(19) United States
(12) Patent Application Publication
(10) Pub. No.: US 2026/0226411 A1
(43) Pub. Date: Aug. 6, 2026

(54) METHODS FOR THE PRODUCTION OF DENDRITIC CELLS BY MODULATING RETINOID SIGNALING

(71) Applicant: FUJIFILM Holdings America Corporation, Valhalla, NY (US)

(72) Inventors: Maksym VODYANYK, Madison, WI (US); Denise ALLARD TROUT, Madison, WI (US); Kasey Nicole DAVIS, Madison, WI (US); Xin ZHANG, Madison, WI (US); Tasi Jo McFARLANE, Madison, WI (US); Christopher McMAHON, Madison, WI (US)

(73) Assignee: FUJIFILM Holdings America Corporation, Valhalla, NY (US)

(21) Appl. No.: 19/531,414

(22) Filed: Feb. 5, 2026

Related U.S. Application Data
(60) Provisional application No. 63/754,378, filed on Feb. 5, 2025.

(51) Int. Cl. C12N 5/0784; C07K 14/525; C07K 14/54; C07K 14/74; C12N 5/00; C12N 5/0783

(52) U.S. Cl. CPC C12N 5/0639; C07K 14/525; C07K 14/5412; C07K 14/70539; C12N 5/0018; C12N 5/0068; C12N 5/0637; C12N 2500/02; C12N 2501/02; C12N 2501/125; C12N 2501/22; C12N 2501/2301; C12N 2501/2306; C12N 2501/25; C12N 2501/385; C12N 2501/42; C12N 2501/52; C12N 2502/1114; C12N 2506/45; C12N 2533/52

(57) ABSTRACT

Provided herein are methods for producing type 2 myeloid dendritic cells from hematopoietic progenitor cells (HPCs), such as induced pluripotent stem cell-derived HPCs, by induction with a retinoid signaling agonist, such as vitamin A.

### PRIORITY CLAIM

This application claims benefit of priority to U.S. Provisional Application Ser. No. 63/754,378, filed Feb. 5, 2025, the entire contents of which are hereby incorporated by reference.

### BACKGROUND

### 1. Field

The present disclosure relates generally to the field of cellular biology. More particularly, it concerns the production of dendritic cells (DCs), such as from induced pluripotent stem cells (iPSCs).

### 2. Description of Related Art

Dendritic cells (DC) represent a diverse group of antigen-presenting cells (APCs) that play a key role in the initiation and regulation of adaptive immune responses. By ontogenetic, morphologic, phenotypic and gene expression criteria, DCs are divided into 3 main groups: conventional DCs (cDC) of myeloid origin, plasmacytoid DCs (pDC) of lymphomyeloid origin, and monocyte derived DCs (moDC). The latter are mostly produced and characterized in vitro, and their presence and role in vivo remain controversial.

cDCs represent a “classic” DC type possessing all functional DC attributes such as sensing, capturing, transferring, and presenting exogeneous and endogenous antigens to T cells in lymphoid organs. Like T cells that are divided into two main subsets (CD8 and CD4) that mediate adaptive immune response to intracellular and extracellular antigens in HLA class I (CD8) and class II (CD4) restricted manners, cDCs include two functionally distinct types including type 1 (cDC1) and type 2 (cDC2) subsets, which are responsible for antigen presentation to CD8 and CD4 T cells, respectively. Also, like more diverse and numerous CD4 T cells, cDC2 constitute the major cDC subset initiating a wide range of CD4 T cell-mediated responses (e.g., Th1, Th2, Th17, Treg). Based on variations in phenotype, gene expression and functions, additional cDC subsets have been postulated (DC3-5), but their origin and relation to known DC or monocytic lineages are poorly defined. A distinguishing feature of cDCs is a high replenishment rate. Another type of DCs include mucosal DCs which reside in the gastrointestinal, respiratory, and reproductive tracts that bridge innate and adaptive immunity.

CD1c is a surface marker expressed on dendritic cell subsets in humans, and is involved in the presentation of lipid-based antigens to T cells. CD14 is a surface marker primarily associated with monocytes and macrophages and expressed at lower level on neutrophils and dendritic cell subsets that is a co-receptor for the detection of bacterial lipopolysaccharides. The CD1c+/CD14− phenotype represents a major type 2 subset of conventional dendritic cells, i.e. the cDC2 population. Dendritic cells are short lived (e.g., <14 days), they migrate and reside briefly for antigen sampling in tissues and then for antigen presentation in lymphoid organs.

Because of their central role in the initiation of immune responses, DCs have a tremendous potential for immunotherapy and vaccination; however, the paucity and post-mitotic state of circulating DCs pose a significant challenge to obtain primary DCs for therapeutic purposes. A seminal finding that peripheral blood monocytes can be efficiently differentiated into DC-like cells (moDC) in vitro in the presence of GM-CSF and IL-4 led to widespread use of this approach to generate DCs for in vitro studies and eventually therapeutic applications. However, the fact that moDCs are pro-inflammatory, poorly migrating and generally inferior to cDCs in antigen-presenting function may explain the limited success of DC therapeutic trials. Therefore, alternative methods for efficient cDC production are needed and should be developed.

Nowadays, iPSCs are widely seen as an alternative and unlimited source of therapeutic cells produced in well-controlled, defined conditions. Human induced pluripotent stem cells (iPSCs) are widely seen as an alternative and unlimited source of therapeutic cells produced in well-controlled, defined conditions. iPSCs offer a limitless source of cDCs but no conditions for such differentiation have been identified thus far, which are serum-free and feeder-free which would be important for developing cell therapies to treat human disease. Numerous earlier reports have demonstrated the potential of human iPSCs to generate DCs. However, most of these studies explored procedures for DC derivation from monocytes (e.g., using GM-CSF and IL-4), and as result, iPSC-derived DCs possess features more resembling macrophages than classical cDCs. Thus, a lack of well-defined conditions of cDC differentiation in vitro limits progress in the iPSC field as well, and although iPSCs offer a unique opportunity to recapitulate normal developmental conditions, no such method has been reported for cDCs to date.

### SUMMARY

Provided herein are methods for producing conventional type 2 dendritic cells (cDC2) from hematopoietic progenitor cells (HPCs) comprising: (a) obtaining a population of HPCs; and (b) differentiating the HPCs in media comprising a retinoid signaling agonist (e.g., in an amount sufficient to induce differentiation of HPCs to DCs) to produce a population of cDC2. The methods may comprise (a) obtaining a population of CD34⁺CD45⁺ HPCs; and (b) differentiating the CD34⁺CD45⁺ HPCs in media comprising a retinoid signaling agonist to produce a population of cDC2. The media may be further defined as dendritic cell differentiation media.

The population of HPCs may comprise cells positive for CD34, CD43, and/or CD45. In some aspects, the population of HPCs is a population of CD34⁺CD45⁺ HPCs, CD34⁺CD43⁺ HPCs, or CD34⁺CD43⁺CD45⁺ HPCs. The population of CD34⁺CD45⁺ HPCs may comprise at least 20%, 30%, 40%, 50%, 60%, 70%, 80%, 90%, 95%, 96%, 97%, 98%, or 99% CD45-positive cells. The population of CD34⁺CD45⁺ HPCs may comprise at least 20%, 30%, 40%, 50%, 60%, 70%, 80%, 90%, 95%, 96%, 97%, 98%, or 99% CD34-positive cells. The population of CD34⁺CD45⁺ HPCs may be a pure population of CD34⁺CD45⁺ HPCs. The population of CD34⁺CD43⁺ HPCs may comprise at least 20%, 30%, 40%, 50%, 60%, 70%, 80%, 90%, 95%, 96%, 97%, 98%, or 99% CD43-positive cells. The population of CD34⁺CD43⁺ HPCs may comprise at least 20%, 30%, 40%, 50%, 60%, 70%, 80%, 90%, 95%, 96%, 97%, 98%, or 99% CD34-positive cells. The population of CD34⁺CD43⁺ HPCs may be a pure population of CD34⁺CD43⁺ HPCs.

In some aspects, the differentiating is further in the presence of a Notch activator, such as a Notch activator is selected from the group consisting of Delta-like ligand 4 (DLL4), Delta-like ligand 1 (DLL1), Jagged 1 (JAG1), and Jagged 2 (JAG2). The Notch activator may be DLL4. The Notch activator may be DLL1. In certain aspects, the media of step (b) further comprises at least one pro-inflammatory cytokine and/or dendritic cell growth factor. For example, the at least one pro-inflammatory cytokine is selected from the group consisting of tumor necrosis factor (TNF), lymphotoxin, IL-33, IL-1β, IL-1α, and IL-33. In specific aspects, the at least one pro-inflammatory cytokine is TNF or lymphotoxin (e.g., 0.5-5 ng/mL, such as 2 ng/mL). In certain aspects, the at least one dendritic cell growth factor is selected from the group consisting of Granulocyte-macrophage colony-stimulating factor (GM-CSF), IL-3, and Macrophage colony-stimulating factor (M-CSF or CSF-1). In particular aspects, the at least one dendritic cell growth factor is GM-CSF. For example, media comprises TNF and GM-CSF.

The retinoid signaling agonist may be a naturally occurring biologically active retinoid or a synthetic agonist of retinoic acid receptor (RAR). In some aspects, the retinoid is vitamin A, such as retinyl acetate, retinol, retinyl palmitate, all-trans retinoic acid, or 9-cis retinoic acid, at a concentration of 1 μM to 5 μM (e.g., 1 μM to 3 μM, or 3 μM to 5 μM). In particular aspects, the vitamin A is retinyl acetate, such as at a concentration of 1 μM to 5 μM (e.g., 1 μM to 2.5 μM, or 2.5 μM to 5 μM) particularly at a concentration of 1 μM to 3 μM. In some aspects, the retinoid signaling agonist is not a RXR-specific agonist. In certain aspects, the retinoid signaling agonist is a RAR-specific agonist, such as a pan-RAR specific agonist or RARα specific agonist.

Exemplary retinoids include but are not limited to retinyl acetate, retinyl palmitate, all-trans retinoic acid (ATRA), 9-cis retinoic acid, or a synthetic agonist of retinoid signaling pathway, such as a pan-RAR (e.g., TTNPB (4-[(E)-2-(5,6,7,8-Tetrahydro-5,5,8,8-tetramethyl-2-naphthalenyl)-1-propenyl]benzoic Acid)) or RARα selective agonist (e.g., AM580 (4-[(5,6,7,8-Tetrahydro-5,5,8,8-tetramethyl-2-naphthalenyl)carboxamido]benzoic acid)). In some aspects, the retinoid signaling agonist is not a RXR-specific agonist (e.g., CD3254). In certain aspects, the pan-RAR or RARα agonist is used in the media at a concentration of about 1-100 nM, such as about 1-30 nM, such as 1-5 nM, 5-10 nM, 1-10 nM, 10-15 nM, 15-20 nM, 20-25 nM, 25-30 nM or 10-30 nM.

In some aspects, the media of step (b) further comprises at least one hematopoietic growth factor. In certain aspects, the at least one hematopoietic growth factor is selected from the group consisting of stem cell factor (SCF), FMS-related tyrosine kinase 3 ligand (FLT3L), and Thrombopoietin (TPO). In particular aspects, the at least one hematopoietic growth factor is SCF.

In some aspects, differentiating comprises culturing HPC in the presence of a Notch activator, pro-inflammatory cytokine, dendritic cell growth factor, and/or hematopoietic growth factor. For example, step (b) comprises differentiating HPCs (e.g., CD34⁺CD45⁺ HPCs) in media comprising vitamin A, a Notch activator (e.g., DLL4), GM-CSF, TNF, and SCF to produce a population of cDC2.

In particular aspects, the population of cDC2 comprises at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) CD1c⁺/CD14⁻ cells. In some aspects, the at least 90% CD1c⁺/CD14⁻ have not undergone a purification step. In certain aspects, the CD1c⁺/CD14⁻ cells express HLA class II (HLA-II) without activation. In particular aspects, the cDC2 are cDC2A, such as C-type lectin domain family 4 member A (CLEC4A)^{high}C-type lectin domain family 10 member A (CLEC10A)⁻ (CLEC4A^{high}CLEC10A⁻) cDC2A (e.g., at least 70%, 75%, 80%, 85%, 86%, 87%, 88%, 89%, 90%, 91%, 92%, 93%, 94%, 95%, 96%, 97%, 98%, or 99% of the cDC2 express CLEC4A and/or less than 15%, 14%, 13%, 12%, 11%, 10%, 9%, 8%, 7%, 6%, 5%, 4%, or 3% of the cDC2 express CLEC10A). In particular aspects, the population of cDC2 comprises at least 85% CLEC4A positive cells and less than 3% CLECL10A positive cells.

In certain aspects, the differentiating is performed on an extracellular matrix-coated surface. In some aspects, the extracellular matrix is selected from the group consisting of fibronectin, a fragment of fibronectin, a 63-kD fragment of recombinant human fibronectin fragment (e.g., rFN-CH-296, also known as RETRONECTIN®), or vitronectin. In some aspects, the differentiating is performed under hypoxic conditions.

In particular aspects, the CD1c⁺/CD14⁻ cDC2 comprise small, semi-adherent cells with multiple dendrites. In specific aspects, the method produces more than 10, 15, 20, 25, 30, 35, 40, 45, 50, 55, 60, 65, 70, 75, or 80 cDC2 per input HPC. In some aspects, the method produces more than 25 cDC2 per input HPC. In particular aspects, the method produces more than 50 cDC2 per input HPC (e.g., 60, 70, 80, 90, or 100 cDC2 per input HPC). The input HPC may be an iPSC-derived HPC or a primary HPC.

In some aspects, the HPCs, such as CD34⁺CD45⁺ HPCs, CD34⁺CD43⁺ HPCs, or CD34⁺CD43⁺CD45⁺ HPCs, are induced pluripotent stem cells (iPSC)-derived HPCs or primary HPCs. In some aspects, the iPSC are T cell-derived iPSC (TiPSC). In particular aspects, obtaining the HPCs, such as CD34⁺CD45⁺ HPCs, CD34⁺CD43⁺ HPCs, or CD34⁺CD43⁺CD45⁺ HPCs, comprises differentiating iPSCs to HPCs, such as CD34⁺CD45⁺ HPCs, CD34⁺CD43⁺ HPCs, or CD34⁺CD43⁺CD45⁺ HPCs. In some aspects, the method produces more than 2,000 cDC2 per input iPSC (e.g., 2,500, 3,000, 3,500, or 4,000 cDC2 per input iPSC). In some aspects, the primary HPCs are derived from cord blood, peripheral blood, or bone marrow. In some aspects, the HPCs are primary cord blood CD34⁺ HPCs.

The CD1c⁺/CD14⁻ cDC2 may further express one or more of CD11c, HLA class II, CD80, CD86, CD197, CD172a/SIRPα, CLEC4A, and CD11b. In some aspects, the CD1c⁺/CD14⁻ cDC2 further express CD11c, HLA class II, CD80, CD86, CD197, CD172a/SIRPα, CLEC4A, and CD11b. In some aspects, the CD1c⁺/CD14⁻ cDC2 further express one or more of Interferon regulatory factor 4 (IRF4) and CLEC10A. In certain aspects, the CD1c⁺/CD14⁻ cDC2 have increased expression of IRF4, CD1C, Integrin subunit alpha X (ITGAX, also known as CD11c), CLEC4A and/or CLEC10A as compared to the HPCs. In some aspects, the CD1c⁺/CD14⁻ cDC2 do not express CLEC9A, Cell Adhesion Molecule 1 (CADM1), X-C motif chemokine receptor 1 (XCR1), Transcription Factor 4 (TCF4), B-cell lymphoma/leukemia 11A (BCL11A), and/or C-Type Lectin Domain Family 4 Member C (CLECL4C). In certain aspects, the CD1c⁺/CD14⁻ cDC2 do not have increased expression of Interferon regulatory factor 8 (IRF8), thrombomodulin (THBD), TCF4, BCL11A, Neuropilin 1 (NRP1), and/or CCAAT/enhancer-binding protein alpha (CEBPA) as compared to the HPCs. In some aspects, the CD1c⁺/CD14⁻ cDC2 have upregulated expression of toll like receptors (TLR) TLR2, TLR7, and TLR10 as compared to the HPCs. In certain aspects, the CD1c⁺/CD14⁻ cDC2 express TLR1, TLR4, TLR6, and TLR8.

In particular aspects, the CD1c⁺/CD14⁻ cDC2 do not express CD303, XCR1, DC-SIGN, CD14, CD88, or CD89. In some aspects, the CD1c⁺/CD14⁻ cDC2 are non-activated resting cDC and do not express CD83.

In some aspects, step (b) produces the population of cDC2 in less than 7 days, such as 6 days. In some aspects, the method further comprises activating the population of cDC2 to acquire a mature phenotype. In particular aspects, activating comprises exposing the population of cDC2 to pro-inflammatory factors, CD40 ligand (CD40L) and/or toll-like receptor (TLR) ligands. For example, the pro-inflammatory factors comprise TNF, IL-1β, IL-6, and/or PGE2. In some aspects, the mature phenotype comprises expression of CD83 and CD197/CCR7. In certain aspects, the mature phenotype comprises increased expression of HLA class II and co-stimulatory receptors CD80, CD86, and CD40 as compared to the non-activated resting cDC2.

In some aspects, the cDC2 trigger proliferation (i.e., stimulation) of T cells in a co-culture with T cells. In certain aspects, the co-culture comprises cDC2 and T cells at a ratio of 1:5 to 1:1280 (e.g., 1:1 to 1:20, 1:5 to 1:50, 1:5 to 1:100, 1:5 to 1:250, 1:5 to 1:320, 1:5 to 500, 1:5 to 1:1000, 1:10 to 1:50, 1:20 to 1:50, 1:30 to 1:50, 1:40 to 1:100, 1:50 to 1:250, 1:100 to 1:300, 1:250 to 1:500, or 1:500 to 1:1000). In certain aspects, the co-culture comprises cDC2 and T cells at a ratio of 1:5 to 1:320. In certain aspects, the activated cDC2 produce IL-23, IL-6 and/or TNF. In some aspects, the activated cDC2 do not produce IL-12p70 or IFN. In certain aspects, the T cells are CD8⁺ T cells and/or CD4⁺ T cells. In certain aspects, the proliferation of T cells is HLA Class II and antigen specific proliferation. In some aspects, the proliferation of T cells is an HLA class II restricted antigen-specific response.

In some aspects, the cDC2 are allogenic. In other aspects, the cDC2 are autologous. In some aspects, the media of step (b) does not comprise or is essentially free of IL-4 and/or IL-13. In particular aspects, the method is performed under serum-free and feeder-free conditions. In some aspects, the method is performed under xeno-free conditions.

Provided herein are compositions comprising at least 80% (e.g., at least 81%, 82%, 83%, 84%, 85%, 85%, 87%, 88%, 89%, 90%, 91%, 92%, 93%, 94%, 95%, 96%, 97%, 98%, or 99%) CD1c⁺/CD14⁻ cDC2 produced by the methods as provided herein.

In some aspects, the composition comprises at least 90% CD1c⁺/CD14⁻ cDC2. In some aspects, the cDC2 further express HLA class II. In certain aspects, the CD1c⁺/CD14⁻ cDC2 further express CD11c, HLA class II, CD80, CD86, CD172a/SIRPα, CLEC4A, and CD11b. In certain aspects, the CD1c⁺/CD14⁻ cDC2 do not express CD303, CD141, XCR1, DC-SIGN, CD14, CD88, or CD89. In some aspects, the cDC2 have been activated to produce mature cDC2. In particular aspects, the mature cDC2 express CD83 and have an increased expression of HLA class II, CCR7, CD80, CD86, and CD40 as compared to the non-activated cDC2.

In some aspects, the CD1c⁺/CD14⁻ cDC2 further express one or more of IRF4 and CLEC10A. In certain aspects, the CD1c⁺/CD14⁻ cDC2 have increased expression of IRF4, CD1C, ITGAX, CLEC4A and/or CLEC10A as compared to the HPCs. In some aspects, the CD1c⁺/CD14⁻ cDC2 do not express CLEC9A, CADM1, XCR1, TCF4, BCL11A, and/or CLECL4C. In certain aspects, the CD1c⁺/CD14⁻ cDC2 do not have increased expression of IRF8, THBD, TCF4, BCL11A, NRP1, and/or CEBPA as compared to the HPCs. In some aspects, the CD1c⁺/CD14⁻ cDC2 have upregulated expression of TLR2, TLR7, and TLR10 as compared to the HPCs. In certain aspects, the CD1c⁺/CD14⁻ cDC2 express TLR1, TLR4, TLR6, and TLR8.

Provided herein are methods of stimulating T cells comprising culturing said T cells with a composition of cDC2 as provided herein. In some aspects, the T cells are CD4⁺ T cells and/or CD8⁺ T cells. In some aspects, T cells and cDC2 are cultured at a ratio of 1:5 to 1:1280 (e.g., 1:1 to 1:20, 1:5 to 1:50, 1:5 to 1:100, 1:5 to 1:250, 1:5 to 1:320, 1:5 to 500, 1:5 to 1:1000, 1:10 to 1:50, 1:20 to 1:50, 1:30 to 1:50, 1:40 to 1:100, 1:50 to 1:250, 1:100 to 1:300, 1:250 to 1:500, or 1:500 to 1:1000). In certain aspects, the culturing further comprises an antigenic peptide. In some aspects, the cDC2 present HLA Class II-bound peptide for antigen-specific stimulation of T cells. In some aspects, the T cells are iPSC-derived T cells. The cells may be autologous or allogeneic.

Provided herein are methods for the generation of a cell population from input human pluripotent stem cells, wherein the cell population comprises CD1c+ cells and is produced under serum-free and feeder-free conditions.

In some aspects, the cell population is produced under xeno-free conditions. In some aspects, the cell population comprises CD14− cells. In certain aspects, more than 30% (e.g., at least 40%, 50%, 60%, 70%, 80%, 90% or 95%) of the cell population is CD1c+. In certain aspects, more than 30% (e.g., at least 40%, 50%, 60%, 70%, 80%, 90% or 95%) of the cell population is CD14−. In some aspects, more than 30% (e.g., at least 40%, 50%, 60%, 70%, 80%, 90% or 95%) of the cell population is CD1c+/CD14−. In particular aspects, the method results in an increase in the percentage of cells expressing CD1c. In some aspects, the increase in the percentage of cells expressing CD1c is induced by retinoid addition.

In certain aspects, the cell population comprises CD1c+ HLA class II+ cells. In particular aspects, more than 30% (e.g., at least 40%, 50%, 60%, 70%, 80%, 90% or 95%) of the cells in the cell population are CD1c+ HLA class II+. In particular aspects, retinoids are used in the generation of the cell population. In some aspects, a Notch activator is used in the generation of the cell population. In particular aspects, a proinflammatory cytokine is used in the generation of the cell population. In some aspects, a DC growth factor is used in the generation of the cell population. In certain aspects, hematopoietic growth factors are used in the generation of the cell population. In some aspects, GM-CSF is used in the generation of the cell population. In some aspects, TNF is used in the generation of the cell population. In specific aspects, exogeneous IL-4 is not added during the generation of the cell population, and wherein the generation of the final cell population occurs in culture media that is essentially free of IL-4.

Provided herein are cell populations generated from input pluripotent stem cells, wherein the cell population comprises CD1c+ cells and is produced under serum-free and feeder-free conditions.

In some aspects, more than 30% (e.g., at least 40%, 50%, 60%, 70%, 80%, 90% or 95%) of the cell population is CD1c+/CD14−. In particular aspects, more than 30% (e.g., at least 40%, 50%, 60%, 70%, 80%, 90% or 95%) of the cells in the cell population are CD1c+ HLA class II+.

In particular aspects, retinoids are used in the generation of the cell population. In some aspects, a Notch activator is used in the generation of the cell population. In particular aspects, a proinflammatory cytokine is used in the generation of the cell population. In some aspects, a DC growth factor is used in the generation of the cell population. In certain aspects, hematopoietic growth factors are used in the generation of the cell population. In some aspects, GM-CSF is used in the generation of the cell population. In some aspects, TNF is used in the generation of the cell population. In specific aspects, exogeneous IL-4 is not added during the generation of the cell population, and wherein the generation of the final cell population occurs in culture media that is essentially free of IL-4.

Provided herein are methods for increasing CD1c expression in a cell population generated from input human pluripotent stem cells, wherein the cell population is CD14⁻. In some aspects, the cell population is produced under serum-free and feeder-free conditions.

Provided herein are methods for increasing CD1c and HLA class II expression in a starting cell population generated from input human pluripotent stem cells, wherein the starting cell population was CD1c− and HLA class II−. In particular aspects, the cell population is produced under serum-free and feeder-free conditions.

Other objects, features and advantages of the present disclosure will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1: Schematic for cDC2 generation from human iPSCs. iPSCs maintained on vitronectin-coated plates in E8 medium were dissociated at 50% confluence after 10 min incubation in 0.5 mM EDTA/PBS, counted and plated into ultra-low attachment flasks (ULA, Corning) at 5×10⁴ cells/mL in hematopoietic differentiation medium 1 (HDM1) supplemented with 1 μM H1152 ROCK inhibitor (day 0). Flasks were placed on the orbital shaker set on 25-30 rpm for uniform size aggregate formation and following culture agitation throughout HPC differentiation. 80% medium was changed every day. Cultures were processed through (1) mesoderm (day 1-2), (2) hemogenic endothelial cell (EC, day 3-5) and (3) budding/floating hematopoietic progenitor cell (HPC, day 6-8) stages in HDM basal medium (see composition in Table 1) supplemented with stage-specific inductive factors as indicated, designated as HDM1, HDM2 and HDM3, respectively. On day 8, CD34⁺CD43⁺CD45^{+/-} HPCs were collected from 30 μM filtrate of entire culture by centrifugation, counted and plated on the Notch ligand (L)-coated plates (DLL1-Fc, DLL4-Fc, JAG1-Fc or JAG2-Fc in the 0.1-1 μg/cm² range) at 5×10³/cm² density for 1-week cDC2 differentiation in DCDM (see composition in Table 2) supplemented with Vitamin A as water-soluble cyclodextrin-loaded retinyl acetate (RLA, Sigma) and indicated DC supportive cytokines (SCF, TPO, FLT3L, GM-CSF, and TNF). cDC2 cultures were maintained in 5% O₂/CO₂ hypoxic incubator. 50% medium was changed every 2^{nd} day. cDC2 emerge as a homogeneous (e.g., >90%) population of CD1c+CD14−HLA class II+ cells on days 6-7 of culture in DCDM. For harvesting, cDC2 were suspended and collected by gentle pipetting. Remaining attached cDC2 could be collected after 10 min incubation in 0.5 mM EDTA/PBS.

FIG. 2: Vitamin A determines cDC2 fate. HPCs were cultured 7 days in DCDM supplemented with DC supportive cytokines (e.g., SCF, TPO, FLT3L, GM-CSF, TNF) without (control) or with Vitamin A (e.g., RLA). Vitamin A was added at a concentration normally detected in human serum (i.e., 2 μM). While mostly CD14+ monocytic cells were detected in control cultures, adding Vitamin A resulted in a near complete suppression of CD14+ cells and development of a more than 90% pure CD1c+CD14− population. In contrast to CD14+ cells, emerging CD1c+CD14− cells express HLA class II (HLA-II) at high levels (e.g., over 90%) without activation—a feature of cDCs. These data highlight distinct differentiation pathways for monocytic and cDC lineages and identify Vitamin A as a determining differentiation factor for the cDC2 lineage. Quadrant value: Without Vitamin A (RLA): CD14−/CD1c−=28.2%; CD14−/CD1c+=4.87%; CD14+/CD1c−=54.0%; CD14+/CD1c+=12.9%. With Vitamin A (RLA): CD14−/CD1c−=8.79%; CD14−/CD1c+=90.8%; CD14+/CD1c−=0.064%; CD14+/CD1c+=0.38%. Without Vitamin A (RLA): HLA-II−/CD1c−=75.1%; HLA-II−/CD1c+=9.02%; HLA-II+/CD1c−=7.10%; HLA-II+/CD1c+=8.79%. With Vitamin A (RLA): HLA-II−/CD1c−=5.88%; HLA-II−/CD1c+=0.46%; HLA-II+/CD1c−=2.97%; HLA-II+/CD1c+=90.7%.

FIG. 3: Dendritic cell morphology of Vitamin A-induced CD1c+CD14− cells. After 6 days of differentiation on RETRONECTIN®-coated plates, CD1c+CD14− cells emerge as morphologically homogenous population of small semi-adherent cells with multiple well-developed dendrites (200× magnification).

FIGS. 4A-4B: Notch signaling strongly promotes Vitamin A-induced cDC2 differentiation. HPCs were cultured 7 days on plates coated (+) or not (−) with Notch ligand (Notch L: DLL4-Fc, 0.25 μg/cm²) in DCDM supplemented with Vitamin A (retinyl acetate at 2 μM) and HPC growth factors only (STF: SCF, TPO, FLT3L) or with TNF and GM-CSF added to STF either alone or in combination (TNF & GM-CSF). cDC2 (CD1c+HLA class II+) phenotyping (FIG. 4A) showed that both TNF and GM-CSF were needed to induce efficient cDC2 differentiation without Notch activation, whereas HPC growth factors (STF) were sufficient to generate highly enriched cDC2 in Notch L+ conditions. Thus, Notch activation replaces the requirement for TNF and GM-CSF to induce cDC2 in the presence of Vitamin A. Nevertheless, both TNF and GM-CSF positively contributed to cDC2 development in Notch L+ conditions. Quantitative cDC2 yields (FIG. 4B) showed that GM-CSF greatly promotes, and TNF additionally contributes to cDC2 growth in Notch L+ cultures. Moreover, TNF significantly up-regulates HLA class II expression by cDC2 (FIG. 4A). Resulting conditions for the most efficient cDC2 generation include all tested components: Vitamin A, Notch L, TNF and GM-CSF. With this procedure, more than 90% pure cDC2 were generated with yields of more than 50 cDC2 per HPC. Considering the more than 50 HPC yields from iPSC, overall efficiency of cDC2 production from iPSC is about 2500 per iPSC in 15 days. It is important to note that in the absence of Vitamin A, no substantial cDC2 production was detected in any conditions, with or without Notch activation (see FIG. 2).

FIG. 5: mRNA profiling confirms the highly selective cDC2 differentiation. HPCs from two iPSC lines (line 1 and line 2) were differentiated 7 days on plates coated or not with Notch ligand DLL4-Fc (0.125 μg/cm²) in DCDM supplemented with Vitamin A, SCF, TPO, FLT3L, GM-CSF and TNF cytokines, referred to as “TNF” and “TNF+DLL4” conditions for 7 day DC differentiation. Total RNA was extracted and analyzed by Illumina RNA sequencing. Selected markers representative of specific DC and macrophage lineages (e.g., cDC2 markers IRF4, CD1C, ITGAX, CLEC10A, and CLEC4A) revealed the highly selective differentiation to cDC2 lineage in both TNF and TNF+DLL4 conditions. No inducible and/or consistent expression of cDC1 (e.g., IRF8, CLEC9A, CADM1, XCR1, and THBD), plasmacytoid DC (pDC) (e.g., TCF4, BCL11A, CLECL4C, NRP1, and IL3RA) or macrophage (Mφ) (e.g., CEBPA, CD14, C5AR1, FCAR, and CD163) markers further confirmed that the differentiated CD1c+CD14− cells most closely resemble cDC2-type dendritic cells.

FIG. 6: CD1c+ cell phenotyping confirms cDC2 identity. Although CD1c is a defining marker of human cDC2 lineage in the absence of monocytic markers (e.g., CD14), iPSC derived CD1c+ cells were tested for expression of additional DC type defining markers by flow cytometry using validated antibodies and comparing to staining with Ig isotype negative controls. The majority of the population of CD1c+ cells also expressed common DC (e.g., CD11c) and APC markers (e.g., HLA class II, CD80, CD86) along with markers associated with the cDC2 lineage (e.g., CD172a/SIRPα, CLEC4A, CD11b). Conversely, the CD1c+ cells do not express markers associated with plasmacytoid DC (e.g., CD303), cDC1 (e.g., CD141, XCR1), monocyte-derived DC (e.g., CD209/DC-SIGN) or monocytes/macrophages (e.g., CD14, CD88, CD89). Thus, the cDC2 identity of iPSC derived CD1c+ cells generated by the present methods was confirmed.

FIGS. 7A-7B: Evaluation of cDC2 inductive factors. cDC2 differentiation was tested in conditions with individual inductive factor withdrawal from the STF/TNF/GM-CSF/RLA-supplemented DCDM medium in both Notch L− and Notch L+ (0.125 μg/cm² DLL4-Fc) cultures. Vitamin A (e.g., retinyl acetate, RLA), SCF and GM-CSF were needed for efficient cDC2 differentiation in both Notch L− and Notch L+ cultures, with their removal resulting in significant decreases in cDC2 cell purity (FIG. 7A) and/or yields (FIG. 7B). TNF was only needed for the Notch L− cultures, but also improved cDC2 purity in Notch L+ cultures. However, the contributions of FLT3L and TPO were minor and not statistically significant. *p<0.05 as compared to respective control “All” values. (FIG. 7A) Expression of HLA-II and CD1c via flow cytometry is shown for the different conditions. (FIG. 7B) Process yield, expressed as number of cDC2 cells produced per input HPC.

FIG. 8: Testing different retinoids revealed a key role of RARα signaling in cDC2 differentiation. cDC2 differentiation was tested in the presence of different agonists of the retinoid signaling pathway in both Notch L− and Notch L+ (DLL4-Fc, 0.125 μg/cm²) cultures. The cDC2-inductive effect of Vitamin A (e.g., retinyl acetate, RLA) could be reproduced by natural (e.g., all-trans retinoic acid, ATRA), synthetic (e.g., TTNPB) pan-RAR and RARα selective (e.g., AM580) agonists, but not RXR-specific CD3254. This indicates that Vitamin A-mediated cDC2 differentiation can be attributed to RAR (specifically RARα) signaling. Notably, Vitamin A is effective and optimal at physiological doses in the 1-3 μM range and toxic at higher concentrations, while tested pan-RAR and RARα agonists were effective at 1000× lower concentrations and had wider optimal ranges (e.g., 1-10+ nM).

FIGS. 9A-9B: TNF and lymphotoxin are optimal cDC2 inductive pro-inflammatory cytokines. Pro-inflammatory cytokine TNF is needed for cDC2 differentiation in cultures without Notch activation. Other pro-inflammatory cytokines were tested in both Notch L− and Notch L+ cultures (DLL4-Fc, 0.125 μg/cm²). Among the tested factors, lymphotoxin (LT), IL-1α, IL-1β and IL-33 all have a similar cDC2 inductive effect in Notch L− cultures and improve cDC2 purity in Notch L+ cultures (FIG. 9A). Concentration titrations demonstrated that maximal cDC2 inductive effect can be achieved at lower TNF and LT doses (1 ng/mL) as compared to IL-1α/β (3 ng/mL) and IL-33 (10 ng/mL). At lower doses, TNF and LT induce more efficient cDC2 differentiation in Notch L− cultures and improve cDC2 purity while not affecting yields in Notch L+ cultures, whereas IL-1α/β and IL-33 are less efficient and decrease cDC2 yields in Notch L+ cultures (FIG. 9B). Nevertheless, although TNF and LT are optimal cDC2 supportive factors, IL-1α, IL-1β and IL-33 can also support cDC2 differentiation. *p<0.05 as compared to respective control “none” values.

FIGS. 10A-10B: Role of different Notch ligands. Most data for Notch L+ cultures were obtained with DLL4 Notch ligand. Other available recombinant Notch L-Fc chimera proteins (DLL1, JAG1 and JAG2) were tested and found supportive for cDC2 differentiation as well. As expected, different Notch ligands show different efficiencies with respect to cDC2 yields, which also depends on their absorption to plastic with or without RETRONECTIN®. In cultures without RETRONECTIN® (FIG. 10A), cDC2 supportive activity of Notch ligands was distributed in the following order: JAG2>DLL1=DLL4>>JAG1, while in cultures with RETRONECTIN® (FIG. 10B), activity of DLL1 and especially DLL4 was significantly higher and resulted in the different hierarchy: DLL4>DLL1>JAG2>>JAG1. Unexpectedly, JAG2 was found superior in conditions without RETRONECTIN®. DLL1 and DLL4 have similar efficiency, albeit much stronger DLL4 activity in presence of RETRONECTIN®. JAG1 activity was much lower in both conditions. Nevertheless, all tested Notch ligands can support cDC2 differentiation when used in optimal concentrations.

FIGS. 11A-11B: Role of DC growth and differentiation factors. GM-CSF is a commonly used factor to promote DC growth and differentiation. IL-3 is a reported alternative factor, though less efficient. M-CSF is commonly used for monocyte/macrophage growth and differentiation, not for DCs. It is remarkable that under cDC2 differentiation conditions with Vitamin A, both IL-3 and M-CSF support cDC2 growth and differentiation. IL-3 alone is less efficient than GM-CSF, however, it may improve cDC2 generation when combined with GM-CSF. M-CSF alone supports highly efficient cDC2 differentiation only in Notch L+ conditions, but in combination with GM-CSF, results in ~2× higher efficiency of cDC2 generation in both Notch L− and Notch L+ cultures. These data show that GM-CSF, IL-3 and M-CSF can support cDC2 growth and differentiation, and their combinations may provide additional advantage to improve purity and/or yields. (FIG. 11A) Flow cytometry of CD14 and CD1c is shown for the different conditions. (FIG. 11B) Process yield, expressed as number of cDC2 cells produced per input HPC.

FIGS. 12A-12B: Suppressive IL-4 effects on cDC2 differentiation. IL-4 is commonly used to generate DCs from primary monocytes in combination with GM-CSF. It is also included in most published methods for DC generation from iPSCs. However, when added to Vitamin A-induced cDC2 differentiation cultures, IL-4 (10 ng/mL) strongly suppressed cDC2 growth and compromises cDC2 purity in Notch L− cultures. Another functionally related cytokine—IL-13 had a similar suppressive effect. (FIG. 12A) Flow cytometry of CD14 and CD1c is shown for the different conditions. (FIG. 12B) Process yield, expressed as number of cDC2 cells produced per input HPC.

FIG. 13: iPSC-derived cDC2 acquire a mature/activated phenotype after short-term exposure to pathogen-associated or pro-inflammatory factors. cDC2 cells generated in 6-day differentiation culture were collected, washed, and replated at 2.5×10⁵/mL density in DCDM supplemented with SCF, FLT3L and GM-CSF at 50 ng/mL each either alone (medium) or with added activators: (1) DC-activating pro-inflammatory cocktail (50 ng/mL TNF, 10 ng/mL IL1β, 10 ng/mL IL6, and 1 μM PGE2); (2) 25 μg/mL particulate zymosan (yeast β-glucan/protein complexes, PAMP ligand for TLR2/Dectin-1 PRR); and (3) 50 ng/mL soluble CD40 ligand (sCD40L, active trimer). After 24-hour incubation, expression of DC maturation/activation marker CD83, HLA class II (HLA-II), co-stimulatory receptors (CD80, CD86, CD40) and homing (CD197/CCR7) receptors was determined. While in control cultures cDC2 cells remain quiescent (CD83−), they become CD83+ and strongly up-regulate expression of HLA class II, co-stimulatory CD80, CD86, CD40 and homing CD197/CCR7 molecules in all activation cultures. Inducible CD197/CCR7 expression indicates migratory potential of iPSC-derived cDC2 cells after activation, a characteristic feature of DCs that is important for homing to secondary lymphoid organs for antigen presentation to resident T cells. Thus, cDC2 cells generated by the established procedure are functionally competent to quickly respond and acquire a mature phenotype after exposure to DC-activating factors. Histogram gates show % positive cells relative to isotype Ig control. Values in italic show mean fluorescence intensity (MFI) relative to isotype Ig control (specific Ab MFI/isotype Ig control MFI).

FIGS. 14A-14B: cDC2 cells can be efficiently differentiated from iPSC reprogrammed from PBMC other than T cells. Much of the data shown included cDC2 differentiated from iPSC lines derived from T cells (“TiPSC” lines). To confirm that cDC2 could be derived from standard iPSC lines, the cDC2 differentiation protocol was performed on four iPSC lines that had been reprogrammed from CD34+ PBMC (Lines K, L and H) or expanded PBMC (Line 101) that were not T cells or B cells (“non-TiPSC”, confirmed by sequencing of the non-rearranged TCR and BCR loci of the iPSC lines). (FIG. 14A) Flow cytometry demonstrated that all lines efficiently differentiated to cDC2 dendritic cells (CD1c+CD14−HLA Class II+). CD83 is not or barely expressed, thus confirming immature non-activated cDC2 differentiation from various iPSC lines. (FIG. 14B) Similar to TiPSC-derived cDC2 cells, the procedure yields large numbers of iPSC-derived cDC2 across lines, particularly in the presence of Notch ligand (DLL4-Fc).

FIGS. 15A-15B: Demonstration of cDC2 phagocytosis and protein processing function by cDC2 cells. cDC2 cells were differentiated from iPSC-derived HPCs for 7 days using vitamin A and TNF on a DLL4-Fc-coated surface, and then either not activated (“None”) or activated with soluble CD40 ligand (“sCD40L”) or TNF/IL1b/IL-6/Prostaglandin E2 (“TB6P”) for 24 hours. (FIG. 15A) The endocytic activity was assessed by measuring the uptake of the fluorescent reporter DQ-ovalbumin (receptor-mediated endocytosis). iPSC-derived cDC2 cells were incubated with 10 μg/10⁵ cells DQ-Ovalbumin at 0° C. or 37° C. for 15 minutes. The fluorescence of ovalbumin labeled with BODIPY FL dye (DQ-Ovalbumin) is self-quenched until the ovalbumin is taken up via the mannose receptor and degraded by endolysosomal proteases. Flow histogram gates showed % positive cells at 37° C. relative to 0° C. The mean fluorescence intensities (MFI) for the different fluorescent cell populations are indicated in each histogram. Non activated cDC2 showed the strongest receptor mediated endocytosis. (FIG. 15B) To measure phagocytosis, the cDC2 cells were incubated with 10 μg/ml pHrodo Green Zymosan A Bioparticles Conjugate that is fluorescent in the low pH environment of lysosomes and endosomes. As expected, phagocytosis is greatly reduced if the 1-hour incubation is carried out at 0° C., or if the cells are pretreated with the specific inhibitor cytochalasin D (Cyto D, 10 μM) for 15 minutes prior to addition of the Bioparticles. Consistent with the literature on primary dendritic cells, non-activated (“immature”) cDC2 showed the strongest phagocytosis.

FIGS. 16A-16B: cDC2-specific TLR expression and functional response. Different DC types can be distinguished by characteristic expression of toll-like receptors (TLR). While cDC1 and pDC express TLR3 and TLR9 in a restricted manner, cDC2 cells express a wide range of other TLRs with highest expression of TLR2. (FIG. 16A) mRNA sequencing of cells differentiated from two iPSC lines (line 1 and line 2) demonstrated low level TLR gene expression in HPCs (TLR1,2,4,6-8,10), which was maintained or up-regulated (TLR2,7,10) in the differentiated cDC2 cells. (FIG. 16B) When cDC2 cells were treated with a panel of TLR-specific ligands (InvivoGen), cDC2 cells responded to TLR2,4 and 7/8 ligands as measured by IL-6 production and up-regulation of activation markers (CD83, HLA-II, CD80, CD86, CD40) and did not have a detectable response to TLR3,9 ligands. This TLR responsiveness profile is characteristic of cDC2 cells. IL-6 was determined by Luminex assay (R&D Systems) and expressed as ng/10⁶ cDC2 cells produced in a 32-hour culture. Expression of activation markers was measured by flow cytometry mean fluorescence intensity (MFI) compared to non-activated control (MFI in activated culture/MFI in non-activated control). NT—not tested.

FIGS. 17A-17B: iPSC-derived cDC2 dendritic cells support allogenic proliferation of primary T cells. cDC2 cells were differentiated from iPSC-derived HPCs for 7 days using the conditions with vitamin A and TNF on DLL4-Fc-coated surface, and then either not activated (“None”) or activated with soluble CD40 Ligand (“sCD40L”) or TNF/IL-1β/IL-6/Prostaglandin E2 (“TB6P”) for 24 hours. The cDC2 cells were washed three times prior to co-culture to remove activating compounds. Allogenic primary T cells were isolated from PBMCs using the Dynabeads Untouched Human T cell Kit to >90% purity (2.4% CD19+, <1% CD14+, <1% CD56+). The T cells were labeled with CellTrace CFSE to track proliferation. 105 T cells/well were co-cultured with decreasing ratios of allogenic cDC2 cells in Primary T cell Medium containing IL-2 (2 ng/ml) for 1 week. (FIG. 17A) No significant T cell proliferation is observed when allogenic cDC2 cells are not added (“medium”), while strong proliferation is triggered with anti-CD3/CD28 antibodies (“anti-CD3/28”), as seen by the diluted CFSE fluorescent intensity in divided T cells. Co-culture with allogenic cDC2 cells triggers robust proliferation of T cells (both CD4+ and CD8+ T cells). The response is particularly potent using TB6P-activated cDC2 cells, where high levels of proliferation are seen at a DC/T cell ratio as low as 1:320 (approximately 300 cDC2 cells per well). (FIG. 17B) Enumeration of the T cells after 1 week of co-culture quantifies the level of expansion. The most potent T cell stimulation is achieved using TB6P-activated cDC2 cells, which trigger a 4-fold expansion at a 1:320 ratio, and up to 10-fold expansion at higher ratios.

FIGS. 18A-18B Derivation of antigen-specific TiPSC-derived T cells that recognize Influenza HA (306-318) peptide in the context of HLA DRB1*01:01. (FIG. 18A) PBMC from an HLA DRB1*01:01+ donor were repeatedly stimulated with Influenza HA (306-318) peptide and autologous feeder cells until a population of peptide-specific CD4+ T cells was detectable using a fluorophore-labeled HLA DRB1*01:01/HA (306-318) tetramer reagent (“DR:HA Tetramer”). No staining is detected using a DRB1*01:01 tetramer bearing an irrelevant control peptide (DR:CLIP Tetramer). The DR:HA Tetramer+/CD4+ population was sorted using FACS, resulting in a >99% population of antigen-specific CD4+ T cells. (FIG. 18B) The antigen-specific T cells were reprogrammed to T cell-derived iPSC clones (“TiPSC”) using a Sendai virus-based reprogramming kit. TiPSC clones were screened for those with TCR rearrangements specific for the HA antigen by differentiating the TiPSC clones to T cells and staining using the DR:HA Tetramer reagent. T cells made from clone 23 (TiPSC line 2) were differentiated for 21 days past the HPC stage and used for cDC2 co-culture experiments. T cells made from this clone express a TCR specific for DRB1*01:01/HA (306-318) as demonstrated by DR:HA Tetramer binding. Expression of CD4 and CD8 on these cells is heterogeneous, with approximately 38% CD4+CD8−, 16% CD8+CD4−, 17% CD4+CD8+, and 29% CD4−CD8−. All CD8+ cells express the CD8αβ heterodimer.

FIGS. 19A-19B: iPSC-derived cDC2 dendritic cells present HLA Class II peptide for antigen-specific stimulation of iPSC-derived T cells. cDC2 cells were differentiated from iPSC-derived HPC for 7 days using vitamin A and TNF on a DLL4-Fc-coated surface, and then either not activated (“None”) or activated with soluble CD40 Ligand (“sCD40L”) or TNF/IL-1β/IL-6/Prostaglandin E2 (“TB6P”) for 24 h. The cDC2 cells were washed three times prior to co-culture to remove activating compounds and pre-incubated with influenza HA (306-318) peptide (“HA peptide”, 1 μg/ml) or irrelevant CLIP peptide (“Control peptide”, 1 μg/ml). Autologous iPSC-derived T cells specific for Influenza HA (306-318) peptide in the context of HLA DRB1*01:01 were differentiated from TiPSC line 2. 2×10⁵ iT cells/well were co-cultured with different ratios of autologous, peptide-loaded cDC2 cells for 1 week. (FIG. 19A) No significant T cell proliferation was observed when cDC2 cells are not added (“medium”), while strong proliferation is triggered with anti-CD3/CD28 antibodies (anti-CD3/28”), as seen by enumeration of the T cells after 1 week of co-culture. Co-culture with autologous cDC2 cells (1:5 DC/T ratio) triggers robust proliferation of iT cells, but only in the presence of HA peptide, demonstrating a Class II-restricted antigen-specific response. Similar iT cell proliferation is triggered by activated or non-activated cDC2 cells. (FIG. 19B) Titration of the DC:T ratio demonstrates reduced antigen-specific T cell proliferation at lower DC:T ratios, but still detectable at 1:320. Unlike what was observed using primary T cells, activated cDC2 cells are not significantly more potent than non-activated cDC2 cells at stimulating iT cells, although slightly better responses were observed using CD40L-activated cDC2 cells.

FIGS. 20A-20B cDC2-specific cytokine production and T cell activation response. (FIG. 20A) Different DC types can be distinguished by characteristic cytokine secretion profile in response to activation stimuli. While cDC1 and pDC produce IL-12p70 and type I IFNα/β, respectively, cDC2 mostly produce pro-inflammatory IL-23, IL-6 and TNF. cDC2 cells generated in 7-day differentiation culture were plated at 2.5×10⁵/mL density in DCDM supplemented with SCF, FLT3L and GM-CSF at 50 ng/mL each either alone (“none”) or with added activators: (1) DC-activating pro-inflammatory cocktail “TB6P” (50 ng/mL TNF, 10 ng/mL IL-1β, 10 ng/mL IL-6, and 1 μM PGE2); (2) 50 ng/mL soluble CD40 ligand (“sCD40L”); and (3) 10 μg/mL particulate zymosan A (yeast β-glucan/protein complexes, PAMP ligand for TLR2/Dectin-1). Culture supernatants were collected after 24-hours. Testing cytokines in supernatants from activated cDC2 cultures revealed a predominant production of IL-23, IL-6 and TNF, but not IL-12p70 or IFNα, thus confirming cDC2-specific cytokine secretion. (FIG. 20B) When cDC2 cells were co-cultured with allogenic peripheral blood CD3+ T cells or autologous influenza hemagglutinin (HA)-specific HLA DRB1-restricted iPSC-derived T cells, the characteristic production of cDC2-specific IL-23, IL-6, TNF was observed. In addition, cytokines associated with Th1 (IFNγ), Th17 (IL-17A), Th2 (IL-4) and cytotoxic T cell (GzmA) responses were detected, thus indicating a profound multifunctional T cell stimulatory activity by cDC2. Specific response in cDC2 co-cultures with autologous HA-specific T cells only in presence of HA antigen peptide also confirms the HLA class II-restricted APC function in cDC2. To avoid interference with the cytokine assays, IL-2 was not added to the co-culture media. Cytokines were measured by multiplex Luminex and LEGENDplex assays (R&D Systems, BioLegend) and expressed as pg per 10⁶ cDC2 in 24 hours (A) or pg per 10⁶ T cells plated (B). cDC2 were added to T cells at 1/5 ratio. N/A—not applicable, nd—not detectable (below assay detection limit).

FIGS. 21A-21C: Stimulation of T cells by iPSC-derived cDC2 cells compared to primary dendritic cells. Monocyte-derived dendritic cells (“moDCs”) were derived from PBMC by isolating monocytes with EasySep human monocytes Enrichment without CD16 depletion kit from Stemcell Technologies, followed by culturing 7 days in DCBM supplemented with GM-CSF (100 ng/ml) and IL-4 (50 ng/ml). cDC2 cells were differentiated from a TiPSC line (reprogrammed from the same donor PBMCs used to make the moDCs) using the conditions previously described with vitamin A and TNF on a DLL4-Fc-coated surface. cDC2 cells and moDC were either non-activated or activated with TNF/IL-1β/IL-6/prostaglandin E2 (“With Activation”) for 24 hours and washed three times prior to co-culture to remove activating compounds. (FIG. 21A) Primary monocyte-derived DCs were CD209 (DC-SIGN)+, HLA class II+, CD11c+, CD1c^{low}, and CD14−. CD83 was strongly upregulated upon activation. Markers such as CD209 distinguish the moDCs as phenotypically different from the TiPSC-derived cDC2 cells. (FIG. 21B) Allogenic primary T cells were isolated from PBMCs using EasySep human T cells isolation Kit from Stemcell Technologies to >90% purity. 2×10⁵ T cells were co-cultured in different ratios with allogenic TiPSC-cDC2 or moDCs for 6 days in T cell differentiation medium (TCDM) containing 2 ng/mL IL-2. T cell proliferation was measured as the total CD3+, CD4+, or CD8+ T cells number fold change compared to the starting population. All DCs stimulated robust T cell proliferation, with more potent stimulation from activated DCs. T cell stimulation by activated cDC2 was slightly superior to primary moDC. T cells without DCs (“None”), with anti-CD3/CD28 stimulating antibodies (0.5 μg/mL each) were included as controls. (FIG. 21C) The same co-culture conditions were conducted at T:DC ratio of 5:1, with the exception that IL-2 was omitted from the TCDM. In the absence of IL-2, the TiPSC-derived cDC2 cells were more potent than primary MoDC at allogenic stimulation of primary T cells, particularly CD4+ T cells. T cells without DC (“None”), with anti-CD3/CD28 stimulating antibodies (0.5 μg/mL each) were included as controls. Anti-CD3/CD28 stimulation of T cells was poor in the absence of IL-2.

FIGS. 22A-22B: cDC2 could be efficiently differentiated from primary cord blood CD34+ HPCs using combination of cDC2 inductive factors Vitamin A, Notch ligand and TNF. (FIG. 22A) The identified cDC2 differentiation factors (e.g., Vitamin A, Notch L, and TNF) were tested to induce cDC2 from primary cord blood CD34+ HPCs. With the SCF, TPO, FLT3L, GM-CSF (STFG) basal condition, neither Vitamin A (RLA), TNF, or Notch L (DLL4) alone was sufficient to induce efficient cDC2 differentiation. The combination of Vitamin A (RLA) and Notch L (DLL4) had the highest suppression of CD14+ cells and direct differentiation to cDC2 lineage. Adding TNF resulted in ~90% pure CD1c+CD14− cDC2. (FIG. 22B) Substantial cDC2 expansion in cultures was also observed with Vitamin A and Notch L combined and only slightly increased with added TNF. In contrast to iPSC-derived cDC2 cells, Notch activation was indispensable for cDC2 differentiation from primary HPCs. In addition, Vitamin A was needed, and TNF was an essential co-factor to produce primary cDC2 with high purity and yields.

FIGS. 23A-23C: cDC2 differentiation potential of iPSC-derived and primary CD34+ HPCs. cDC2 differentiation in iPSC-derived and primary CD34+ HPCs of embryonic (cord blood) and adult (peripheral blood, bone-marrow) origin was compared using combination of Notch L (DLL4-Fc, 0.25 μg/cm²), Vitamin A (water-soluble retinyl acetate, 2 μM, Sigma) and TNF (2 ng/mL) as cDC2 inductive factors. (FIG. 23A) High purity CD1c+CD14− cDC2 cultures were derived from all HPC types. Lower CD1c expression was observed in adult HPC cultures, especially in those isolated from peripheral blood, however, >95% cells in all cultures expressed HLA class II (HLA-II) at high levels, suggesting cDC2 identity of CD1c^{low} cells. (FIG. 23B) In terms of cDC2 yields, remarkably similar high yield proliferative cDC2 cultures were obtained with iPSC-derived and cord blood HPCs, while cDC2 yields in cultures from adult HPCs were ~6× lower due to much lower frequency of cDC2 differentiation initiating cell clusters in culture (visual observation). Nevertheless, after 6-day cDC2 differentiation, all cultures contained a morphologically identical population of small semi-adherent cells (100× magnification) (FIG. 23C).

FIG. 24: Notch activation drives cDC2 differentiation towards cDC2A type. The cDC2 lineage includes two ontogenically and functionally divergent subsets: potent T cell stimulatory cDC2A and pro-inflammatory cDC2B, which can be distinguished by CLEC4A^{high}CLEC10A− and CLEC4A^{low}CLEC10A+ phenotypes, respectively. cDC2 differentiated 7 days in DCDM on plates coated (Notch L+) or not (Notch L−) with DLL4-Fc (0.25 μg/cm2) were assessed for CLEC4A and CLEC10A expression. While in the Notch L− cultures cDC2 cells express both CLEC4A and CLEC10A at low levels (consistent with cDC2B type), cDC2 cells differentiated in the Notch L+ cultures up-regulate CLEC4A and express minimal or undetectable CLEC10A, suggesting that Notch activation directs cDC2 differentiation towards the cDC2A subset. Histogram gates show percentage positive cells relative to isotype Ig control. Values in italics show the ratio of mean fluorescence intensity (MFI) relative to isotype Ig control (specific Ab MFI/isotype Ig control MFI).

### DETAILED DESCRIPTION

Provided herein are methods for producing cDC2 CD1c+/CD14− cells from iPSC-derived HPCs. The present methods can result in the rapid and efficient generation of more than 80% (e.g., 81%, 82%, 83%, 84%, 85%, 86%, 87%, 88%, 89%, 90%, 91%, 92%, 93%, 94%, 95%, 96%, 97%, 98%, 99%, or 100%) pure cDC2 from iPSCs with unprecedented yields of more than 50 cDC2 cells per input HPC in about 1 week, and more than 2000 cDCs per iPSC in about 2 weeks. The method was developed in conditions compatible with clinical cell manufacturing including being serum-free, xeno-free, and feeder cell-free, and can be translated to therapeutic grade cDC2 production at a large scale.

Previous methods for producing dendritic cells have included culturing differentiating cells, such as iPSCs or primary cells, on feeder cells. This could involve culturing differentiating cells fully or partly on feeder cells. Thus, any cDC2-containing cell population produced by feeder cells would not be feeder-free and would introduce complexity and risks for therapeutic use. Additionally, one common feeder cell type is OP9 stromal mouse cells cultured in serum-containing media, which results in a cDC2-containing cell population that is not xeno-free or serum-free, introducing the risk of adventitious viruses.

Other methods use antibodies to purify the desired cell population (e.g., cDC2 cells) from a cell population at the end of a differentiating process. These antibodies may be produced from rodent immortalized hybridomas, and cells to be purified are exposed to antibodies during purification (e.g., magnetically-activated cell sorting or fluorescence-activated cell sorting) producing a cell preparation that is not xeno-free and may contain residual antibodies or magnetic beads.

A cell population produced under serum-free, and/or feeder-free conditions is important as regulatory agencies like the EMA (European Medicines Agency) and the FDA (U.S. Food and Drug Administration) rigorously regulate therapies intended for infusion into human patients. For safety reasons, these regulatory agencies require extensive (and expensive) risk mitigation for any experimental drug product that has been exposed to serum, xeno-origin reagents or feeder cells during manufacturing of the therapy intended for infusion into human patients.

Generation of a final cell population from input human pluripotent stem cells, wherein some of the cells in the final cell population are one or more of CD1c+, CD14− and HLA class II+ comprises first providing a population of pluripotent stem cells in vitro, and culturing under conditions sufficient to drive the pluripotent cells to differentiate. The pluripotent stem cells are differentiated to HPCs which are then differentiated to induce the emergence of a cell population wherein some of the cells are CD1c+ and/or CD14− and/or some of the cells in the final cell population are CD1c+ and/or HLA class II+. Some of the cells being CD1c+, CD14− and/or HLA class II+ means a non-zero percentage of the cells express these cell marker patterns. These percentages may be at least 10%, at least 20%, at least 30%, at least 40%, at least 50%, at least 60%, at least 80%, or at least 90%. The pluripotent stem cells may be induced pluripotent stem cells or embryonic stem cells. The present methods may generate CD1c+CD14− cells from human induced pluripotent stem cells under serum-free (i.e., free of animal serum) and feeder-free conditions. The conditions may also be xeno-free.

Although the role of retinoids, Notch signaling and pro-inflammatory cytokines in cDC2 development in vivo was demonstrated in mice more than 10 years ago, no methods for in vitro cDC2 derivation based on these inductive factors have been reported, either from primary or iPSC-derived cell sources. Here, the inventors developed a method for the rapid and efficient generation of cDC2 from iPSC or primary HPCs, which can provide an unlimited source of pure cDC2 for in vitro studies and therapeutic applications. It was found that Vitamin A is an important factor in determining cDC2 fate, but only in specific supportive conditions, which include either Notch activation, or the pro-inflammatory factor TNF combined with the DC growth factor GM-CSF. The most efficient cDC2 production was achieved by combining all components: Vitamin A, Notch activator, TNF and GM-CSF. Efficient cDC2 generation may also be achieved in the continuous presence of hematopoietic growth factor SCF, optimally in combination with TPO and FLT3L. The procedure can produce the cDC2 in about 6-7 days when starting from iPSC-derived hematopoietic progenitor cells (HPCs) to generate an at least 80% pure population of cDC2 with an efficiency of at least 50 cDC2 produced per single HPC. The purity and efficiency of the present methods far surpass any reported DC derivation procedure from iPSCs. With average HPC yields of at least 50 (e.g., at least 55, 60, 70, or 75) cDC2 produced per iPSC, the overall efficiency of cDC2 production is more than 2,500 per single iPSC in as little as 14-15 days.

cDC2 were identified by the specific CD1c+HLA class II+CD14− phenotype and cDC2 identity was confirmed by extended phenotyping: CD1c+ cells express common DC (CD11c) and APC markers (HLA class II, CD80, CD86) along with cDC2 markers (CD172a/SIRPα, CLEC4a, and CD11b), but lack expression of pDC (CD303), cDC1 (CD141 and XCR1), moDC (CD209/DC-SIGN), and monocyte/macrophage (CD14, CD88, and CD89) markers. Morphologically, cDC2 represent a homogeneous population of small, semi-adherent cells with long dendrites, distinct from previously reported iPSC-derived DCs, which are large, heteromorphic cells resembling macrophages.

DCs sense pathogens through a repertoire of pattern recognition receptors (PRR) evolutionally conserved to recognize a wide range of pathogen-associated molecular patterns (PAMP) ligands. Upon binding PAMP ligands and/or within the pro-inflammatory milieu, DCs become activated to perform antigen-presenting function. DC activation is accompanied by up-regulation of HLA and co-stimulatory molecules, and expression of specific markers of activated or so-called “mature” DCs, such as CD83. Consistent with the non-activated quiescent state, iPSC-derived cDC2 were found to express HLA class II and low-to-moderate levels of co-stimulatory (CD80, CD86, CD40) molecules, but not CD83. However, after only 1 day of exposure to TLR2/Dectin-1 PAMP ligand zymosan or a combination of DC-activating pro-inflammatory factors (TNF, IL-1β, IL-6, PGE2), cDC2 strongly up-regulated HLA class II, CD80, CD86, CD40, and became CD83+. The rapid response of iPSC-derived cDC2 to known DC activators demonstrates their functional competence and highlights an important advantage of the present procedure to generate non-activated, quiescent cDC2 capable of functional response to environmental pathogens/antigen challenge or pro-inflammatory conditions.

The present methods demonstrated that the inclusion of Vitamin A (e.g., retinyl acetate), not only induces cDC2, but also completely suppresses the development of monocytic cells, thus generating “true” cDC2, free of monocyte-like features, and eliminating any contamination with DC-like monocyte derivatives. The studies also demonstrated that IL-4, which is commonly used for DC generation in vitro, has a negative effect on Vitamin A-induced cDC2 generation. By adding IL-4, which is a common practice and used in previously reported DC derivation procedures from iPSCs, no similar results could be achieved. An additional advantage of the present methods is that they can be serum-, feeder-, and xeno-free cell culture conditions compatible with clinical cell manufacturing (e.g., CTS “cell therapy systems” grade media/supplements or similar), so they can be translated to large-scale therapeutic grade production.

These methods were developed for the rapid and efficient production of pure cDC2 from iPSCs in conditions compatible with clinical cell manufacturing, without serum, feeder cells, or xenogenic components. These methods are a straightforward process with culture seeding and feeding, and one culture filtration step for HPC isolation, but without costly cell enrichment or purification procedures. It may be used for large-scale production as well as cDC2 manufacturing for in vitro studies, drug screening, or cell therapy. The cell therapy may be allogeneic, partially HLA-matched, or autologous.

The present cDC2 may be used for the stimulation of T cells, such as antigen-specific stimulation of T cells (e.g., iPSC-derived T cells or primary T cells).

### I. Definitions

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

The term “exogenous,” when used in relation to a protein, gene, nucleic acid, or polynucleotide in a cell or organism refers to a protein, gene, nucleic acid, or polynucleotide that has been introduced into the cell or organism by artificial or natural means; or in relation to a cell, the term refers to a cell that was isolated and subsequently introduced to other cells or to an organism by artificial or natural means. An exogenous nucleic acid may be from a different organism or cell, or it may be one or more additional copies of a nucleic acid that occurs naturally within the organism or cell. An exogenous cell may be from a different organism, or it may be from the same organism. By way of a non-limiting example, an exogenous nucleic acid is one that is in a chromosomal location different from where it would be in natural cells, or is otherwise flanked by a different nucleic acid sequence than that found in nature.

The term “cell” is herein used in its broadest sense in the art and refers to a living body that is a structural unit of tissue of a multicellular organism, is surrounded by a membrane structure that isolates it from the outside, has the capability of self-replicating, and has genetic information and a mechanism for expressing it. Cells used herein may be naturally-occurring cells or artificially modified cells (e.g., fusion cells, genetically modified cells, etc.).

The term “stem cell” refers herein to a cell that under suitable conditions is capable of differentiating into a diverse range of specialized cell types, while under other suitable conditions is capable of self-renewing and remaining in an essentially undifferentiated pluripotent state. The term “stem cell” also encompasses a pluripotent cell, multipotent cell, precursor cell and progenitor cell. Exemplary human stem cells can be obtained from hematopoietic or mesenchymal stem cells obtained from bone marrow tissue, embryonic stem cells obtained from embryonic tissue, or embryonic germ cells obtained from genital tissue of a fetus. Exemplary pluripotent stem cells can also be produced from somatic cells by reprogramming them to a pluripotent state by the expression of certain transcription factors associated with pluripotency; these cells are called “induced pluripotent stem cells” or “iPSCs or iPS cells”.

An “embryonic stem (ES) cell” is an undifferentiated pluripotent cell which is obtained from an embryo in an early stage, such as the inner cell mass at the blastocyst stage, or produced by artificial means (e.g. nuclear transfer) and can give rise to any differentiated cell type in an embryo or an adult, including germ cells (e.g. sperm and eggs).

“Induced pluripotent stem cells (iPScs or iPS cells)” are cells generated by reprogramming a somatic cell by expressing or inducing expression of a combination of factors (herein referred to as reprogramming factors). iPS cells can be generated using fetal, postnatal, newborn, juvenile, or adult somatic cells. Factors that may be used to reprogram somatic cells to pluripotent stem cells include, for example, Oct4 (sometimes referred to as Oct 3/4), Sox2, c-Myc, Klf4, Nanog, and Lin28. Somatic cells may be reprogrammed by expressing at least two reprogramming factors, at least three reprogramming factors, at least four reprogramming factors, at least five reprogramming factors, at least six reprogramming factors, or at least seven reprogramming factors to reprogram a somatic cell to a pluripotent stem cell.

“Pluripotent stem cell” refers to a stem cell that has the potential to differentiate into all cells constituting one or more tissues or organs, or preferably, any of the three germ layers: endoderm (interior stomach lining, gastrointestinal tract, the lungs), mesoderm (muscle, bone, blood, urogenital), or ectoderm (epidermal tissues and nervous system).

As used herein, the term “somatic cell” refers to any cell other than germ cells, such as an egg, a sperm, or the like, which does not directly transfer its DNA to the next generation. Typically, somatic cells have limited or no pluripotency. Somatic cells used herein may be naturally-occurring or genetically modified.

“Differentiation” is the process by which a less specialized cell becomes a more specialized cell type. “Dedifferentiation” is a cellular process in which a partially or terminally differentiated cell reverts to an earlier developmental stage, such as pluripotency or multipotency. “Transdifferentiation” is a process of transforming one differentiated cell type into another differentiated cell type. Typically, transdifferentiation by programming occurs without the cells passing through an intermediate pluripotency stage—i.e., the cells are programmed directly from one differentiated cell type to another differentiated cell type. Under certain conditions, the proportion of progeny with characteristics of the new cell type may be at least about 1%, 5%, 25% or more in order of increasing preference.

As used herein, the term “subject” or “subject in need thereof” refers to a mammal, preferably a human being, male or female at any age that is in need of a cell or tissue transplantation. Typically, the subject is in need of cell or tissue transplantation (also referred to herein as recipient) due to a disorder or a pathological or undesired condition, state, or syndrome, or a physical, morphological or physiological abnormality which is amenable to treatment via cell or tissue transplantation.

A “survival agent” refers to an agent which promotes and/or supports cell survival when added to cell culture media. For example, Rho-associated kinase (ROCK) inhibitors or Myosin II-specific inhibitors may be used as survival agents. In particular aspects, these survival agents promote aggregation of cells in culture.

“Rho-associated kinase inhibitors,” abbreviated as “ROCK inhibitors,” refer to any substance that inhibits or reduces the function of Rho-associated kinase or its signaling pathway in a cell, such as a small molecule, an siRNA, a miRNA, an antisense RNA, or the like. “ROCK signaling pathway,” as used herein, may include any signal processors involved in the ROCK-related signaling pathway, such as the Rho-ROCK-Myosin II signaling pathway, its upstream signaling pathway, or its downstream signaling pathway in a cell. Examples of ROCK inhibitors include, but are not limited to, a Rho-specific inhibitor, a ROCK-specific inhibitor, a MRLC (myosin regulatory light chain)-specific inhibitor, or a Myosin II-specific inhibitor.

As used herein, “administering” shall mean delivering in a manner which is affected or performed using any of the various methods and delivery systems known to those skilled in the art. Administering can be performed, for example, intravenously, orally, via implant, transmucosally, transdermally, intramuscularly, or subcutaneously. Specifically, envisioned is topical administration. “Administering” can also be performed, for example, once, a plurality of times, and/or over one or more extended periods.

“Super donors” are referred to herein as individuals that are homozygous for certain MHC class I and II genes. These homozygous individuals can serve as super donors and their cells, including tissues and other materials comprising their cells, can be transplanted in individuals that are either homozygous or heterozygous for that haplotype. The super donor can be homozygous for the HLA-A, HLA-B, HLA-C, HLA-DR, HLA-DP and/or HLA-DQ locus/loci alleles, respectively.

An “agonist” or “activator” as used herein refers to drug or chemical that binds to a receptor inside a cell or on its surface and causes activation of the receptor to produce a biological response which is the same or similar to the action as the substance that normally binds to the receptor.

A “retinoid signaling agonist” is referred to herein as a compound, such as a small molecule, that activates the retinoid signaling pathway. The agonist may be a synthetic agonist. Exemplary agonists include but are not limited to a naturally occurring biologically active retinoids (e.g., vitamin A including retinol, retinyl acetate, retinyl palmitate, all-trans retinoic acid, and 9-cis retinoic acid), or synthetic agonists of retinoic acid receptors (RARs), alpha, beta gamma or RXR. An RAR agonist is a compound that binds to retinoic acid receptors, leading to the activation of target gene expression by facilitating the exchange between corepressor and coactivator complexes. This results in chromatin alterations and posttranslational modifications that regulate important transcription processes. Other exemplary agonists include TTNPB, AM580, and CD1530.

A “notch activator” as referred to herein a physiological ligand or synthetic compound that activates the Notch signaling pathway through Notch1 or Notch2 receptors, or downstream of the receptors. Exemplary Notch ligand-Fc chimera proteins include but are not limited to DLL4, DLL1, JAG1, and JAG2. The activator may be a synthetic agonist or an engineered activated Notch construct (i.e., a transgenic Notch activator).

A “pro-inflammatory cytokine” as used herein refers to a cytokine that promotes inflammation and is secreted from cells such as immune cells like Th1 cells, CD4+ cells, macrophages, and dendritic cells, or in the present methods added to a cell culture. Cytokines includes interleukins (IL), chemokines, interferons, and tumor necrosis factors (TNF). Exemplary pro-inflammatory cytokines include but are not limited to tumor necrosis factor (TNF; also referred to as TNFα), IL-1β, IL-6, IL-18, IL-33, interferon gamma (IFNγ), lymphotoxin and IL-1α.

“Dendritic cell medium (DCDM)” as used herein refers to a medium which promotes DC differentiation. The DCDM may comprise a retinoid signaling agonist, DC supportive cytokines or growth factors, such as SCF, TPO, FTL3L, GM-CSF, and/or TNF, and a Notch activator. Exemplary components of the DCDM may comprise a defined, serum-free, cGMP-manufactured medium designed for the proliferation and manipulation of human T cells, dendritic cells, and other immune cells (e.g., Cell Therapy Systems (CTS) AIM V w/o phenol red/antibiotics, serum replacement (e.g., CTS* Immune cell SR), GlutaMax, Ascorbic acid 2-phosphate Mg, and 1-Thioglycerol.

A “dendritic cell growth factor” as used herein refers to a compound which promotes DC proliferation, maturation, and/or differentiation. Exemplary DC growth factors include but are not limited to GM-CSF, IL3, M-CSF, IL-7, VEGF, G-CSF, and FLT3L.

An “antigen presenting cell or APC” as used herein refers to a regulator of the immune system to connect innate and adaptive immunity by regulating T cell responses. Examples of APCs include dendritic cells and macrophages.

### II. Pluripotent Stem Cells

Provided herein are methods and compositions for providing cDC (e.g., cDC2) from pluripotent stem cells. The pluripotent stem cells may be stem cells including but are not limited to, induced pluripotent stem cells and embryonic stem cells.

The pluripotent stem cells used herein may be human embryonic stem cells (ESCs) or induced pluripotent stem cells (iPSCs) which are capable of long-term proliferation in vitro, while retaining the potential to differentiate into all cell types of the body, including the cDC of the present disclosure. Thus, these cells could potentially provide an unlimited supply of patient-specific functional cDC for both drug development and therapeutic uses.

### A. Embryonic Stem Cells

In certain aspects, the pluripotent stem cells are embryonic stem cells (ESCs). ES cells are derived from the inner cell mass of blastocysts and have a high in vitro differentiating capability. ES cells can be isolated by removing the outer trophectoderm layer of a developing embryo, then culturing the inner mass cells on a feeder layer of non-growing cells. The replated cells can continue to proliferate and produce new colonies of ES cells which can be removed, dissociated, replated again and allowed to grow. This process of “subculturing” undifferentiated ES cells can be repeated a number of times to produce cell lines containing undifferentiated ES cells (U.S. Pat. Nos. 5,843,780; 6,200,806; 7,029,913). ES cells have the potential to proliferate while maintaining their pluripotency. For example, ES cells are useful in research on cells and on genes which control cell differentiation. The pluripotency of ES cells combined with genetic manipulation and selection can be used for gene analysis studies in vivo via the generation of transgenic, chimeric, and knockout mice.

Methods for producing mouse ES cells are well known. In one method, a preimplantation blastocyst from the 129 strain of mice is treated with mouse antiserum to remove the trophoectoderm, and the inner cell mass is cultured on a feeder cell layer of chemically inactivated mouse embryonic fibroblasts in medium containing fetal calf serum. Colonies of undifferentiated ES cells that develop are subcultured on mouse embryonic fibroblast feeder layers in the presence of fetal calf serum to produce populations of ES cells. In some methods, mouse ES cells can be grown in the absence of a feeder layer by adding the cytokine leukemia inhibitory factor (LIF) to serum-containing culture medium (Smith, 2000). In other methods, mouse ES cells can be grown in serum-free medium in the presence of bone morphogenetic protein and LIF (Ying et al., 2003).

Human ES cells can be produced or derived from a zygote or blastocyst-staged mammalian embryo produced by the fusion of a sperm and egg cell, nuclear transfer, pathogenesis, or the reprogramming of chromatin and subsequent incorporation of the reprogrammed chromatin into a plasma membrane to produce an embryonic cell by previously described methods (Thomson and Marshall, 1998; Reubinoff et al., 2000). In one method, human blastocysts are exposed to anti-human serum, and trophectoderm cells are lysed and removed from the inner cell mass which is cultured on a feeder layer of mouse embryonic fibroblasts. Further, clumps of cells derived from the inner cell mass are chemically or mechanically dissociated, replated, and colonies with undifferentiated morphology are selected by micropipette, dissociated, and replated. In some methods, human ES cells can be grown without serum by culturing the ES cells on a feeder layer of fibroblasts in the presence of basic fibroblast growth factor (Amit et al., 2000). In other methods, human ES cells can be grown without a feeder cell layer by culturing the cells on a protein matrix such as MATRIGEL™ or laminin in the presence of “conditioned” medium containing basic fibroblast growth factor (Xu et al., 2001).

ES cells can also be derived from other organisms including rhesus monkey and marmoset by previously described methods (Thomson and Marshall, 1998; Thomson et al., 1995; Thomson and Odorico, 2000; U.S. Pat. No. 5,843,780), as well as from established mouse and human cell lines. For example, established human ES cell lines include MAOI, MA09, ACT-4, HI, H7, H9, H13, H14 and ACT30. As a further example, mouse ES cell lines that have been established include the CGR8 cell line established from the inner cell mass of the mouse strain 129 embryos, and cultures of CGR8 cells can be grown in the presence of LIF without feeder layers.

ES stem cells can be detected by protein markers including transcription factor Oct4, alkaline phosphatase (AP), stage-specific embryonic antigen SSEA-1, stage-specific embryonic antigen SSEA-3, stage-specific embryonic antigen SSEA-4, transcription factor NANOG, tumor rejection antigen 1-60 (TRA-1-60), tumor rejection antigen 1-81 (TRA-1-81), SOX2, or REX1.

### B. Induced Pluripotent Stem Cells

In other aspects, the pluripotent stem cells used herein are induced pluripotent stem (iPS) cells, commonly abbreviated iPS cells or iPSCs. The induction of pluripotency was originally achieved in 2006 using mouse cells (Yamanaka et al. 2006) and in 2007 using human cells (Yu et al. 2007; Takahashi et al. 2007) by reprogramming of somatic cells via the introduction of transcription factors that are linked to pluripotency. The use of iPSCs circumvents most of the ethical and practical problems associated with large-scale clinical use of ES cells, and patients with iPSC-derived autologous transplants may not require lifelong immunosuppressive treatments to prevent graft rejection.

With the exception of germ cells, any cell can be used as a starting point for iPSCs. For example, cell types could be keratinocytes, fibroblasts, hematopoietic cells, mesenchymal cells, liver cells, or stomach cells. T cells may also be used as a source of somatic cells for reprogramming (U.S. Pat. No. 8,741,648; U.S. Publication No. 2015/0191697). There is no limitation on the degree of cell differentiation or the age of an animal from which cells are collected; even undifferentiated progenitor cells (including somatic stem cells) and finally differentiated mature cells can be used as sources of somatic cells in the methods disclosed herein. iPS cells can be grown under conditions that are known to differentiate human ES cells into specific cell types, and express human ES cell markers including: SSEA-1, SSEA-3, SSEA-4, TRA-1-60, and TRA-1-81.

Somatic cells can be reprogrammed to produce iPS cells using methods known to one of skill in the art. One of skill in the art can readily produce iPS cells, see for example, Published U.S. Patent Application No. 2009/0246875, Published U.S. Patent Application No. 2010/0210014; Published U.S. Patent Application No. 2012/0276636; U.S. Pat. Nos. 8,058,065; 8,129,187; PCT Publication NO. WO 2007/069666 A1, U.S. Pat. Nos. 8,268,620; 8,546,140; 9,175,268; 8,741,648; U.S. Patent Application No. 2011/0104125, and U.S. Pat. No. 8,691,574, which are incorporated herein by reference. Generally, nuclear reprogramming factors are used to produce pluripotent stem cells from a somatic cell. At least three, or at least four, of Klf4, c-Myc, Oct3/4, Sox2, Nanog, and Lin28 may be used. Oct3/4, Sox2, c-Myc and Klf4 may be used. Oct3/4, Sox2, Nanog, and Lin28 may be used.

Mouse and human cDNA sequences of these nuclear reprogramming substances are available with reference to the NCBI accession numbers mentioned in WO 2007/069666 and U.S. Pat. No. 8,183,038, which are incorporated herein by reference. Methods for introducing one or more reprogramming substances, or nucleic acids encoding these reprogramming substances, are known in the art, and disclosed for example, in U.S. Pat. Nos. 8,268,620, 8,691,574, 8,741,648, 8,546,140, in published U.S. Pat. Nos. 8,900,871 and 8,071,369, which are both incorporated herein by reference.

Once derived, iPSCs can be cultured in a medium sufficient to maintain pluripotency. The iPSCs may be used with various media and techniques developed to culture pluripotent stem cells, more specifically, embryonic stem cells, as described in U.S. Pat. No. 7,442,548 and U.S. Patent Pub. No. 2003/0211603. In the case of mouse cells, the culture is carried out with the addition of Leukemia Inhibitory Factor (LIF) as a differentiation suppression factor to an ordinary medium. In the case of human cells, it is desirable that basic fibroblast growth factor (bFGF) be added in place of LIF. Other methods for the culture and maintenance of iPSCs, as would be known to one of skill in the art, may be used with the methods disclosed herein.

Undefined conditions may be used; for example, pluripotent cells may be cultured on fibroblast feeder cells or a medium that has been exposed to fibroblast feeder cells in order to maintain the stem cells in an undifferentiated state. The cell may be cultured in the co-presence of mouse embryonic fibroblasts treated with radiation or an antibiotic to terminate the cell division, as feeder cells. Alternately, pluripotent cells may be cultured and maintained in an essentially undifferentiated state using a defined, feeder-independent culture system, such as a TESR™ medium (Ludwig et al., 2006a; Ludwig et al., 2006b) or E8™/Essential 8™ medium (Chen et al., 2011).

Plasmids have been designed with a number of goals in mind, such as achieving regulated high copy number and avoiding potential causes of plasmid instability in bacteria, and providing means for plasmid selection that are compatible with use in mammalian cells, including human cells. Particular attention has been paid to the dual requirements of plasmids for use in human cells. First, they are suitable for maintenance and fermentation in E. coli, so that large amounts of DNA can be produced and purified. Second, they are safe and suitable for use in human patients and animals. The first requirement calls for high copy number plasmids that can be selected for and stably maintained relatively easily during bacterial fermentation. The second requirement calls for attention to elements such as selectable markers and other coding sequences. Plasmids that encode a marker may be composed of: (1) a high copy number replication origin, (2) a selectable marker, such as, but not limited to, the neo gene for antibiotic selection with kanamycin, (3) transcription termination sequences, including the tyrosinase enhancer and (4) a multicloning site for incorporation of various nucleic acid cassettes; and (5) a nucleic acid sequence encoding a marker operably linked to the tyrosinase promoter. In particular aspects, the plasmids do not comprise a tyrosinase enhancer or promoter. There are numerous plasmid vectors that are known in the art for inducing a nucleic acid encoding a protein. These include, but are not limited to, the vectors disclosed in U.S. Pat. Nos. 6,103,470; 7,598,364; 7,989,425; and 6,416,998, and U.S. application Ser. No. 12/478,154 which are incorporated herein by reference.

An episomal gene delivery system can be a plasmid, an Epstein-Barr virus (EBV)-based episomal vector (U.S. Pat. No. 8,546,140), a yeast-based vector, an adenovirus-based vector, a simian virus 40 (SV40)-based episomal vector, a bovine papilloma virus (BPV)-based vector, or a lentiviral vector. A viral gene delivery system can be an RNA-based or DNA-based viral vector (PCT/JP2009/062911, PCT/JP2011/069588).

### C. Embryonic Stem Cells Derived by Somatic Cell Nuclear Transfer

Pluripotent stem cells for producing the cDC could also be prepared by means of somatic cell nuclear transfer, in which a donor nucleus is transferred into a spindle-free oocyte. Stem cells produced by nuclear transfer are genetically identical to the donor nuclei. In one method, donor fibroblast nuclei from skin fibroblasts of a rhesus macaque are introduced into the cytoplasm of spindle-free, mature metaphase II rhesus macaque ooctyes by electrofusion (Byrne et al., 2007). The fused oocytes are activated by exposure to ionomycin, then incubated until the blastocyst stage. The inner cell mass of selected blastocysts are then cultured to produce embryonic stem cell lines. The embryonic stem cell lines show normal ES cell morphology, express various ES cell markers, and differentiate into multiple cell types both in vitro and in vivo.

### D. MHC Haplotype Matching

Major Histocompatibility Complex is the main cause of immune-rejection of allogeneic organ transplants. There are three major class I MHC haplotypes (A, B, and C) and three major MHC class II haplotypes (DR, DP, and DQ). The HLA loci are highly polymorphic and are distributed over 4 Mb on chromosome 6. The ability to haplotype the HLA genes within the region is clinically important since this region is associated with autoimmune and infectious diseases and the compatibility of HLA haplotypes between donor and recipient can influence the clinical outcomes of transplantation. HLAs corresponding to MHC class I present peptides from inside the cell and HLAs corresponding to MHC class II present antigens from outside of the cell to T-lymphocytes. Incompatibility of MHC haplotypes between the graft and the host triggers an immune response against the graft and leads to its rejection. Thus, a patient can be treated with an immunosuppressant to prevent rejection. HLA-matched stem cell lines may overcome the risk of immune rejection.

Because of the importance of HLA in transplantation, the HLA loci are usually typed by serology and PCR for identifying favorable donor-recipient pairs. Serological detection of HLA class I and II antigens can be accomplished using a complement mediated lymphocytotoxicity test with purified T or B lymphocytes. This procedure is predominantly used for matching HLA-A and -B loci. Molecular-based tissue typing can often be more accurate than serologic testing. Low resolution molecular methods such as SSOP (sequence specific oligonucleotide probes) methods, in which PCR products are tested against a series of oligonucleotide probes, can be used to identify HLA antigens, and currently these methods are the most common methods used for Class II-HLA typing. High resolution techniques such as SSP (sequence specific primer) methods which utilize allele specific primers for PCR amplification can identify specific MHC alleles.

MHC compatibility between a donor and a recipient increases significantly if the donor cells are HLA homozygous, i.e. contain identical alleles for each antigen-presenting protein. Most individuals are heterozygous for MHC class I and II genes, but certain individuals are homozygous for these genes. These homozygous individuals can serve as super donors and grafts generated from their cells can be transplanted in all individuals that are either homozygous or heterozygous for that haplotype. Furthermore, if homozygous donor cells have a haplotype found in high frequency in a population, these cells may have application in transplantation therapies for a large number of individuals.

iPSCs partially matched to the patient may be used for vaccines or tumor antigens for presentation to the patient's immune system. Efficient tumor antigen presentation and stimulatory signals by antigen-presenting cells (APCs) is fundamental for a sustained activation of cytotoxic T and B-cells, and important for the success of any immunotherapeutic approach. Indeed, many tumors present reduced self-antigen presentation due to decreased expression of major histocompatibility complex (MHC)-class 1 molecules, which results from the strong selective pressure imposed on tumor cells in order to evade immunity during early tumorigenesis.

Accordingly, iPSCs of the present methods may be produced from somatic cells of the subject to be treated, or another subject with the same or substantially the same HLA type as that of the patient. In one case, the major HLAs (e.g., the three major loci of HLA-A, HLA-B and HLA-DR) of the donor are identical to the major HLAs of the recipient. In some cases, the somatic cell donor may be a super donor; thus, iPSCs derived from a MHC homozygous super donor may be used to generate cDC. Thus, the cDC derived from a super donor may be transplanted in subjects that are either homozygous or heterozygous for that haplotype. For example, the cDC can be homozygous at two HLA alleles such as HLA-A and HLA-B. As such, cDC produced from super donors can be used in the methods disclosed herein, to produce cDC that can potentially “match” a large number of potential recipients.

The cDC described herein can find use in a broad array of clinical applications involving transplantation of cells and/or tissues. The cDC are HLA compatible with a recipient, and therefore can be introduced into the recipient without the need for immunosuppressive therapy. The cDC can be matched for HLA matching for antigen presentation.

### III. Differentiation to Dendritic Cells

Methods are provided herein for the differentiation of HPCs (e.g., CD34+CD45+ cells or CD34+CD43+ cells) to DCs, such as CD1c⁺CD14⁻ at a high purity and efficiency. The DC may be cDC, such as cDC2 which are HLA-II⁺. The cDC2 may be T cell stimulatory cDC2A (e.g., CLEC4A^{high}CLEC10A−, CD117+, ESAM+, CD7+, and/or CD11b−/low) or pro-inflammatory cDC2B (e.g., CLEC4A^{low}CLEC10A+, CLEC12A+, CX3CR1+, CD209a+, and CD11b+/high). In particular aspects, the cDC2 are cDC2A, such as CLEC4A^{high}CLEC10A− cDC2A. The HPCs may be derived from iPSCs, ESCs, or primary cells. The HPCs may be derived from iPSCs, such as described in U.S. Pat. No. 12,129,486, incorporated herein by reference in its entirety. The iPCS may be derived from any cell type including but not limited to T cells, B cells, CD34+ PBMCs, or expanded PBMCs. The HPCs may be primary cord blood CD34+ HPCs or be derived from peripheral blood or bone marrow.

In certain aspects, differentiating the PSCs to HPCs comprises the sequential steps of culturing or maintaining a plurality of substantially undifferentiated pluripotent cells in a first defined media (e.g., HDM1) which is free or essentially free of IL-3, Flt3 ligand, and GM-CSF, culturing the cells in a second defined media (e.g., HDM2) comprising BMP4 (e.g., 25 ng/mL), FGF2 (e.g., 50 ng/mL), and VEGF (e.g., 50 ng/mL) sufficient to expand or promote differentiation in a plurality of the cells, and culturing the cells in a third defined media (e.g., HDM3) comprising IL-3 and Flt3 ligand, sufficient to expand or promote differentiation in a plurality of the cells. The HDM1 may comprise FGF2 (e.g., 50 ng/mL), VEGF (e.g., 50 ng/mL), a GSK3 inhibitor (e.g., CHIR99021 at 2-3 μM) and may further comprise blebbistatin or a ROCK inhibitor and. The HDM3 may comprise one or more of the cytokines selected from the group consisting of IL-3 (e.g., 20 ng/mL), SCF (e.g., 50 ng/mL), FLT3L (Flt3 ligand at 20 ng/mL) TPO (e.g., 50 ng/mL), and BMP4 (e.g., 5 ng/mL). In some aspects, the HDM comprises heparin. In some aspects, the method comprises culturing the cells at an atmospheric pressure of less than 25% oxygen, such as less than 24%, 23%, 22%, 21%, 20%, 19%, 18%, 17%, 16%, 15%, 10%, or 5% oxygen. In certain aspects, the HPCs express CD34. In particular aspects, the HPCs express at least two markers from the group consisting of CD43, CD34, CD31, CD41, CD235 and CD45.

The iPSCs may be cultured in HDM1 for mesoderm induction (e.g., Day 0 to Day 2, such as about 2 days), followed by culture in HDM2 for hemogenic EC (e.g., Day 2 to Day 5, such as about 3 days), and then cultured in HDM3 (e.g., Day 5 to Day 8, such as about 3 days) for producing HPCs. The HPC differentiation may be performed in ultra low attachment (ULA) flasks in aggregate swirling culture.

The HPCs may then be isolated and differentiated to cDC in dendritic cell differentiation medium (DCDM). Exemplary DCDM may comprise Vitamin A, such as as retinyl acetate (RLA, such as 1-5 μM, such as about 2 μM) and DC supportive cytokines SCF (e.g., 25-100 ng/mL, such as about 50 ng/mL), TPO (e.g., 25-100 ng/mL, such as about 50 ng/mL), FLT3L (e.g., 25-100 ng/mL, such as about 50 ng/mL), GM-CSF (e.g., 25-100 ng/mL, such as about 50 ng/mL), and TNF (e.g., 1-10 ng/mL, such as about 2 ng/mL). The HPCs may be cultured in DCDM for about 5-10 days, such as about 5, 6, 7, 8, or 9 days (e.g., Day 8 to Day 15). The differentiation to cDC may be performed under static conditions, such as on a Notch L substrate, and under hypoxia (e.g., 5% O₂).

The HPCs can be cultured in the presence of a retinoid pathway agonist, such as vitamin A. The purity and efficiency of the differentiation of the HPCs to cDC can be increased by Notch activation, pro-inflammatory cytokines, and/or growth factors. For example, TNF and GM-CSF may be used to enhance differentiation to cDC. In particular, activation of Notch signaling (e.g., DLL4, DLL1, JAG1, and JAG2) can yield a synergistic effect resulting in unprecedented purity and yields. The complete method can be performed in a serum-free and feeder-free system. In particular aspects, the present methods do not comprise embryoid bodies. The present cDC may be differentiated from HPCs as a single cell suspension, such as in static culture on an extracellular protein-coated surface.

In particular aspects, the retinoid signaling agonist is vitamin A. The vitamin A may be in the media at a concentration of about 1-3 μM. The combination of vitamin A, TNF, and GM-CSF can be used to produce a highly pure population of CD1c+CD14−HLA-II+ cells, such as at least 50%, 60%, 70%, 80%, 95%, 96%, 97%, 98%, 99%, or 100%. The combination of vitamin A and a notch ligand can produce a highly pure population of CD1c+CD14−HLA class II+ cells, such as at least 70%, 80%, 90%, 95%, 96%, 97%, 98%, 99%, or 100%. Alternatively, the combination of vitamin A, notch ligand, TNF, and GM-CSF can produce a population of at least 90%, 91%, 92%, 93%, 94%, 95%, 96%, 97%, 98%, 99%, or 100% CD1c+CD14−HLA class II+ cells.

Retinoid signaling agonist(s), such as retinoids, may be used to differentiate the HPCs to cDC to increase the percentage of cells expressing CD1c. Exemplary retinoids include but are not limited to retinyl acetate, retinyl palmitate, all-trans retinoic acid (ATRA), 9-cis retinoic acid, or a synthetic agonist of retinoid signaling pathway, such as a pan-RAR (e.g., TTNPB (4-[(E)-2-(5,6,7,8-Tetrahydro-5,5,8,8-tetramethyl-2-naphthalenyl)-1-propenyl]benzoic Acid)) or RARα selective agonist (e.g., AM580 (4-[(5,6,7,8-Tetrahydro-5,5,8,8-tetramethyl-2-naphthalenyl)carboxamido]benzoic acid)). In some aspects, the retinoid signaling agonist is not a RXR-specific agonist (e.g., CD3254). In some aspects, Vitamin A is used in the media at a concentration of about 1-3 μM. In certain aspects, the pan-RAR or RARα agonist is used in the media at a concentration of about 1-10 nM or 10-100 nM.

Notch activator(s) may be used to differentiate the HPCs to cDC. Exemplary Notch activators include one or more of Notch 1 or Notch 2 ligands DLL1, DLL4, JAG1, JAG2, or a synthetic agonist of Notch signaling pathway. The Notch activator may be a recombinant protein bound to plastic, immobilized to beads, or in solution. In some aspects, the Notch ligand could be expressed by feeder cells.

Proinflammatory cytokines can also be used to induce cDC including but not limited to TNF, IL-1β, lymphotoxin and IL-1α. Additional cytokines that may be used for cDC differentiation include IL-18, IL-33, BMP4, and IFNα. In certain aspects, the method does not comprise the presence of BMP4, IFNα, IL-1, IL-12, and/or CXCL8.

DC growth factors being used in the generation of the final cell population refers to one or more of the growth factors manipulated in the culture conditions used in generation of a final cell population. Examples include GM-CSF and IL-3. Hematopoietic growth factors include but are not limited to SCF alone or in combination with FLT3L and TPO. Additional growth factors may include FGF, VEGF, G-CSF, and/or IL-7.

TABLE 1
Exemplary composition of hematopoietic differentiation medium (HDM).

| # | Component | Concentration | | Vendor, catalog # |
|---|---|---|---|---|
| 1 | IMDM | 50% | | ThermoFisher, 12440053 |
| 2 | Ham's F-12 | 50% | | ThermoFisher, 11765054 |
| 3 | CTS* B27 XenoFree Vitamin A-, 50× | 1× | | ThermoFicher, A5047601 |
| 4 | MEM non-essential amino acids, 100× | 1× | | ThermoFisher, 11140050 |
| 5 | Ascorbic acid 2-phosphate Mg | 250 | μM | Sigma, A8960 |
| 6 | Heparin | 100 | ng/mL | Sigma, H3149 |
| 7 | 1-Thioglycerol | 100 | μM | Sigma, M6145 |

*Cell Therapy Systems (CTS), ThermoFisher TM.

TABLE 2
Exemplary composition of dendritic cell differentiation medium (DCDM).

| # | Component | Concentration | | Vendor, catalog # |
|---|---|---|---|---|
| 1 | CTS* AIMV, w/o phenol red/antibiotics | 95% | | ThermoFisher, A3830801 |
| 2 | CTS* Immune cell SR | 5% | | ThermoFisher, A2596101 |
| 3 | GlutaMax, 100× | 1× | | ThermoFisher, 35050061 |
| 4 | Ascorbic acid 2-phosphate Mg | 250 | μM | Sigma, A8960 |
| 5 | 1-Thioglycerol | 100 | μM | Sigma, M6145 |

*Cell Therapy Systems (CTS), ThermoFisher TM.

The cDC2 may be activated to acquire a mature phenotype. Activating may comprise exposing the population of cDC to pro-inflammatory factors, CD40 ligand (CD40L) and/or toll-like receptor (TLR) ligands. The pro-inflammatory factors may comprise TNF, IL-1β, IL-6, and/or PGE2. In some aspects, the maturation agent is LPS. In some aspects, the maturation agents do not comprise LPS or CpG.

### B. Cell Culture Conditions

The culturing conditions according to the present disclosure will be appropriately defined depending on the medium and stem cells used. The medium according to the present disclosure can be prepared using a medium to be used for culturing animal cells as its basal medium. As the basal medium, any of E8, TeSR, BME, BGJb, CMRL 1066, Glasgow MEM, Improved MEM Zinc Option, IMDM (Iscove's Modified Dulbecco's Medium comprising 4-(2-hydroxyethyl)-1-piperazineethanesulfonic acid (HEPES) buffer, sodium pyruvate, and higher levels of amino acids and vitamins than standard DMEM), Medium 199, Eagle MEM, αMEM, DMEM, Ham, RPMI 1640, and Fischer's media, as well as any combinations thereof can be used, but the medium is not particularly limited thereto as far as it can be used for culturing animal cells.

In particular aspects, the medium according to the present disclosure is a serum-free medium. The serum-free medium refers to media with no unprocessed or unpurified serum and accordingly, can include media with purified blood-derived components or animal tissue-derived components (such as growth factors). The medium according to the present disclosure may contain or may not contain any alternatives to serum. The alternatives to serum can include materials which appropriately contain albumin (such as lipid-rich albumin, albumin substitutes such as recombinant albumin, plant starch, dextrans and protein hydrolysates), transferrin (or other iron transporters), fatty acids, insulin, collagen precursors, trace elements, 2-mercaptoethanol, 3′-thiolglycerol, or equivalents thereto. The alternatives to serum can be prepared by the method disclosed in International Publication No. 98/30679, for example. Alternatively, any commercially available materials can be used for more convenience. The commercially available materials include knockout Serum Replacement (KSR), Immune Cell Serum Replacement (ICSR), Chemically-defined Lipid concentrated (Gibco).

The medium of the present disclosure can also contain fatty acids or lipids, amino acids (such as non-essential amino acids), vitamin(s), growth factors, cytokines, antioxidant substances, 2-mercaptoethanol or 1-thioglycerol, pyruvic acid, buffering agents, and inorganic salts. The concentration of 1-thioglycerol can be, for example, about 0.05 to 1.0 mM, and particularly about 0.1 to 0.5 mM, but the concentration is particularly not limited thereto as long as it is appropriate for culturing the stem cell(s).

A culture vessel used for culturing the stem cell(s) can include, but is particularly not limited to: flask, flask for tissue culture, dish, petri dish, dish for tissue culture, multi dish, micro plate, micro-well plate, multi plate, multi-well plate, micro slide, chamber slide, tube, tray, CellSTACK® Chambers, culture bag, roller bottle, and bioreactors, such as PBS500 and/or PBS3, as long as it is capable of culturing the stem cells therein. The stem cells may be culture in a volume of at least or about 0.2, 0.5, 1, 2, 5, 10, 20, 30, 40, 50 ml, 100 ml, 150 ml, 200 ml, 250 ml, 300 ml, 350 ml, 400 ml, 450 ml, 500 ml, 550 ml, 600 ml, 800 ml, 1000 ml, 1500 ml, 2000 ml, or any range derivable therein, depending on the needs of the culture. The culture vessel may be a bioreactor, which may refer to any device or system that supports a biologically active environment. The bioreactors may have a volume of at least or about 2, 4, 5, 6, 8, 10, 15, 20, 25, 50, 75, 100, 150, 200, 500 liters, 1, 2, 4, 6, 8, 10, 15 cubic meters, or any range derivable therein.

The culture vessel can be cellular adhesive or non-adhesive and selected depending on the purpose. The cellular adhesive culture vessel can be coated with any of substrates for cell adhesion such as extracellular matrix (ECM) to improve the adhesiveness of the vessel surface to the cells. The substrate for cell adhesion can be any material intended to attach stem cells or feeder cells (if used). The substrate for cell adhesion includes collagen, gelatin, poly-L-lysine, poly-D-lysine, laminin, vitronectin and fibronectin and mixtures thereof for example Matrigel™, and lysed cell membrane preparations (Klimanskaya et al., 2005).

Other culturing conditions can be appropriately defined. For example, the culturing temperature can be about 30 to 40° C., for example, at least or about 31, 32, 33, 34, 35, 36, 37, 38, 39° C. but particularly not limited to them. The CO₂ concentration can be about 1 to 10%, for example, about 2 to 5%, or any range derivable therein. The oxygen tension can be at least or about 1, 5, 8, 10, 20%, or any range derivable therein.

The methods of the present disclosure can be also used for a suspension culture of stem cells, including suspension culture on carriers (Fernandes et al., 2007) or gel/biopolymer encapsulation (United States Patent 20070116680). The term suspension culture of the stem cells means that the stem cells are cultured under non-adherent condition with respect to the culture vessel or feeder cells (if used) in a medium. The suspension culture of stem cells includes a dissociation culture of stem cells and an aggregate suspension culture of stem cells. The term dissociation culture of stem cells means that suspended stem cells is cultured, and the dissociation culture of stem cells include those of single stem cell or those of small cell aggregates composed of a plurality of stem cells (for example, about 2 to 400 cells). When the aforementioned dissociation culture is continued, the cultured, dissociated cells form a larger aggregate of stem cells, and thereafter an aggregate suspension culture can be performed. The aggregate suspension culture includes an embryoid culture method (see Keller et al., 1995), and a SFEB method (Watanabe et al., 2005); International Publication No. 2005/123902). The methods of the present disclosure can significantly improve the survival rate and/or differentiation efficiency of stem cells in a suspension culture.

Bioreactors can be grouped according to general categories including: static bioreactors, stirred flask bioreactors, rotating wall vessel bioreactors, hollow fiber bioreactors and direct perfusion bioreactors. Within the bioreactors, cells can be free, or immobilized, seeded on porous 3-dimensional scaffolds (hydrogel). In certain aspects, the bioreactor is a suspension bioreactor for efficient mixing with homogeneous particle suspension and low shear stress.

The methods disclosed utilized herein may use all GMP compatible materials and be scaled to multiple (e.g., 3 L) bioreactor manufacturing batches to yield the purity and cell numbers needed for dendritic cell therapy development.

Serum-free media offer numerous advantages in cell culture, making them valuable for various applications, including therapeutic cell production. These advantages not only enhance the safety and regulatory compliance but also contribute to the reproducibility, scalability, and cost-effectiveness of the cell culture process. Serum-free media can eliminate the risk of introducing contaminants, adventitious agents, or pathogens that may be present in serum. This ensures the safety and purity of cell culture processes, meeting the stringent regulatory requirements for therapeutic cell production. The use of serum-free media aligns with regulatory guidelines for the manufacture of biologics and cell-based therapies. Regulatory agencies recommend or require serum-free or xeno-free culture conditions to minimize the risks associated with serum, ensuring compliance and facilitating the regulatory approval of cell therapy products. Serum-free media can provide a defined and controlled environment, minimizing variability associated with different serum batches, origins, and concentrations. This leads to more consistent and reproducible cell culture conditions, important for obtaining reliable and comparable results in research and large-scale production. Serum-free media can be tailor-made, supplemented with defined growth factors, or specific nutrients that support optimal cell growth and viability. This leads to improved cell expansion, proliferation, and maintenance of desired cell phenotypes, for successful therapeutic cell manufacturing. It can also enable efficient scale-up of cell culture processes in bioreactors or production systems. They provide consistent and reliable cell yields required for large-scale commercial applications, addressing the need for industrialization and commercial viability of therapeutic cell production. Finally, it can potentially reduce manufacturing costs by eliminating the need for expensive serum components. Moreover, the absence of serum simplifies the cell culture process by eliminating steps such as serum heat inactivation or lipid depletion, making it a more cost-effective and straightforward approach.

Xeno-free media provide several advantages in cell culture, particularly for applications involving the production of therapeutic cells These advantages include the enhanced safety, regulatory compliance, consistency, reproducibility, and clinical relevance provided by xeno-free media. Xeno-free media can eliminate the risk of introducing potentially harmful contaminants or adventitious agents derived from animal-derived serum products. This ensures the safety and purity of cell culture processes, meeting the stringent regulatory requirements for therapeutic cell production and minimizing potential adverse effects on patients. The use of xeno-free media aligns with regulatory guidelines for the manufacturing of biologics and cell-based therapies. Regulatory agencies often recommend or require xeno-free or serum-free culture conditions to reduce the risks associated with animal-derived components. By using xeno-free media, cell therapy products can meet regulatory standards, facilitating their approval and commercialization. It can provide a defined and controlled environment, reducing variability associated with lot-to-lot variations in animal-derived serum. This leads to more consistent and reproducible cell culture conditions, important for obtaining reliable and comparable results in research and large-scale production of therapeutic cells. Th xeno-free media allow for better standardization and comparability of cell culture processes. By eliminating the variability introduced by animal-derived serum, xeno-free media can provide a more controlled and consistent environment, leading to robust and standardized manufacturing protocols. This enhances process scalability, reproducibility, and clinical translation of cell therapy products. Xeno-free media can significantly reduce the risk of introducing xenogeneic components that might trigger an immune response or generate adverse reactions in patients receiving cell therapies. By excluding animal-derived materials, xeno-free media minimize immunogenicity concerns and enhance the safety profile of therapeutic cell products. It can facilitate the efficient scale-up of cell culture processes in bioreactors or production systems. They provide consistent and reliable cell growth and expansion, addressing the need for large-scale commercial manufacturing of therapeutic cells. The scalability offered by xeno-free media contributes to the industrialization and wider availability of cell-based therapies. Finally, xeno-free media streamline cell culture processes by eliminating the need for animal-derived serum and associated steps such as serum heat inactivation or lipid depletion. This simplifies and reduces the complexity of the culture protocols, providing a more straightforward and cost-effective approach to therapeutic cell production.

Feeder-free culture can eliminate the need for animal-derived feeder cells, leading to increased experimental reproducibility and standardization. This system allows for higher cell growth rates and reduces the risk of contamination. Feeder cells may contaminate the stem cell population with non-stem cell derivatives, posing challenges for downstream applications. Feeder-free culture enables the isolation of homogenous stem cell populations, reducing the risk of unwanted cellular heterogeneity. Feeder-free culture enables large-scale expansion of stem cells, facilitating the production of ample quantities required for various applications, such as cell therapies and drug discovery. This scalability is important for meeting the growing demand for stem cell-based products. Animal-derived feeder cells may introduce unknown factors that can interfere with the genetic stability of cultured stem cells. Feeder-free culture eliminates this risk, ensuring the safety and integrity of the stem cell population, thus making it suitable for clinical applications. The absence of feeders and undefined components in feeder-free culture allows for greater control over the stem cell environment. This leads to reduced batch-to-batch variability, improving experimental reproducibility and the validity of research findings. Feeder-free culture systems can utilize defined or serum-free media, which enables better control over cell culture conditions. This compatibility with defined media facilitates the standardization of protocols, leading to more consistent research outcomes and easier transition to clinical applications. Feeder-free culture can eliminate the dependence on feeder cell preparation and maintenance, saving time and labor. This simplified workflow increases efficiency and optimizes resource utilization, making it a cost-effective approach for stem cell research and production. The use of feeder-free culture systems reduces the reliance on animal-derived materials, addressing ethical concerns associated with the use of animal products in research. This promotes the development and commercialization of stem cell products with a broader societal acceptance. Feeder-free culture systems contribute to the standardization of stem cell research protocols, enabling better comparison and reproducibility between different laboratories. This advancement helps establish consensus and best practices in the field, fostering collaborations and driving progress in stem cell research.

### C. Characterization of Dendritic Cells

The cells obtained according to the present methods can be characterized according to a number of phenotypic criteria.

Tissue-specific markers can be detected using any suitable immunological technique—such as flow immunocytometry or affinity adsorption for cell-surface markers, immunocytochemistry (for example, of fixed cells or tissue sections) for intracellular or cell-surface markers, Western blot analysis of cellular extracts, and enzyme-linked immunoassay, for cellular extracts or products secreted into the medium. Antibodies that distinguish cDC markers from other DC are available commercially from suppliers like BD Biosciences, BioLegend, and ThermoFisher. Expression of an antigen by a cell is said to be antibody-detectable if a significantly detectable amount of antibody will bind to the antigen in a standard immunocytochemistry or flow cytometry assay, optionally after fixation of the cells, and optionally using a labeled secondary antibody.

cDC2 can be identified by the specific CD1c+HLA class II+CD14− phenotype and cDC2 identity can be confirmed by extended phenotyping using one or more of the following: CD1c+ cells express common DC (CD11c) and APC markers (HLA class II, CD80, CD86) along with cDC2 markers (CD172a/SIRPα, CLEC4a, and CD11b), but lack expression of pDC (CD303), cDC1 (CD141 and XCR1), moDC (CD209/DC-SIGN), and monocyte/macrophage (CD14, CD88, and CD89) markers. Morphologically, cDC2 represent a homogeneous population of small, semi-adherent cells with long dendrites, distinct from previously reported iPSC-derived DCs, which are large, heteromorphic cells resembling macrophages.

Mature, activated cDC2 may express CD83 and CCR7 and have increased expression of HLA class II and co-stimulatory receptors CD80, CD86, and CD40 as compared to the non-activated resting cDC. Other markers that may be assessed include HLA-DR (MHC class II), and CD172a (SIRPα) which are highly expressed by non-activated cDC2. MHC class I may be expressed by cDC2. CLEC9A is cDC1 marker which is generally not expressed by cDC2. CLEC10A is expressed weakly in cDC2, such as cDC2 generated in Notch L− conditions. CD163 is macrophage marker, CD115 (M-CSFR) is expressed on HPCs, CD117 (SCFR) is expressed broadly on hematopoietic cells at a low level, and CD205 is expressed by moDCs and cDC1.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods using publicly available sequence data (GenBank). Expression of tissue-specific markers as detected at the protein or mRNA level is considered positive if the level is at least or about 2-, 3-, 4-, 5-, 6-, 7-, 8-, or 9-fold, and more particularly more than 10-, 20-, 30, 40-, or 50-fold above that of a control cell, such as an undifferentiated pluripotent stem cell or other unrelated cell type.

Once markers have been identified on the surface of cells of the desired phenotype, they can be used for immunoselection to further enrich the population by techniques such as immunopanning or antibody-mediated fluorescence-activated cell sorting.

Where derived from an established line of pluripotent stem cells, the cell populations and isolated cells of the present disclosure can be characterized as having the same genome as the line from which they are derived. This means that the chromosomal DNA will be over 90% identical between the pluripotent stem cells and the cDC, which can be inferred if the cDC are obtained from the undifferentiated line through the course of normal mitotic division. The characteristic that cDC are derived from the parent cell population is important in several respects. In particular, the undifferentiated cell population can be used for producing additional cells with a shared genome—either a further batch cDC, or another cell type that may be useful in therapy.

### IV. Methods of Use

The cDC cells provided by methods and compositions provided herein can be used in a variety of applications. These include but are not limited to transplantation or implantation of the cells in vivo; screening cytotoxic compounds, carcinogens, mutagens growth/regulatory factors, pharmaceutical compounds, etc., in vitro; elucidating the mechanism of diseases and injuries; studying the mechanism by which drugs and/or growth factors operate; diagnosing and monitoring cancer in a patient; gene therapy; and the production of biologically active products.

cDC of the present disclosure may be used for the treatment of cancer, infections, and immune disorders. For example, cDC may be used for vaccination and immunomodulation. The cDC may be administered by injection, such as intravenous or local injection. The cDC may be loaded with an antigen and activated prior to injection to the subject. The cDC may be HLA matching with the patient (e.g., a full or partial match). The cDC may be activated for CCR7 expression and/or loaded with antigens to stimulate T cells, such as with a peptide, protein, or protein expressed by the cells via engineering.

cDC of the present disclosure can be used commercially to screen for factors (such as solvents, small molecule drugs, peptides, oligonucleotides) or environmental conditions (such as culture conditions or manipulation) that affect the characteristics of such cells and their various progeny.

Cytotoxicity can be determined in the first instance by the effect on cell viability, survival, morphology, and the expression of certain markers and receptors. Effects of a drug on chromosomal DNA can be determined by measuring DNA synthesis or repair. [³H]-thymidine or BrdU incorporation, especially at unscheduled times in the cell cycle, or above the level required for cell replication, is consistent with a drug effect. Unwanted effects can also include unusual rates of sister chromatid exchange, determined by metaphase spread. The reader is referred to Vickers (pp 375-410 in In vitro Methods in Pharmaceutical Research, Academic Press, 1997) for further elaboration.

A method of treatment may include treatment of a cancer in a subject. Examples of cancers can include acute lymphoblastic leukemia, acute myeloid leukemia, adrenocortical carcinoma, basal-cell carcinoma, bile duct cancer, bladder cancer, bone tumor, osteosarcoma/malignant fibrous histiocytoma, brainstem glioma, brain tumor, cerebellar astrocytoma, cerebral astrocytoma/malignant glioma, ependymoma, medulloblastoma, supratentorial primitive neuroectodermal tumors, breast cancer, bronchial adenomas/carcinoids, Burkitt's lymphoma, carcinoid tumor, cervical cancer, chronic lymphocytic leukemia, chronic myelogenous leukemia, chronic myeloproliferative disorders, colon cancer, cutaneous T-cell lymphoma, endometrial cancer, ependymoma, esophageal cancer, Ewing's sarcoma, intraocular melanoma, retinoblastoma, gallbladder cancer, gastric cancer, gastrointestinal carcinoid tumor, gastrointestinal stromal tumor, germ cell tumor, glioma, childhood visual pathway and hypothalamic, Hodgkin lymphoma, melanoma, islet cell carcinoma, Kaposi sarcoma, renal cell cancer, laryngeal cancer, leukemia, lymphomas, mesothelioma, neuroblastoma, non-Hodgkin lymphoma, oropharyngeal cancer, osteosarcoma, ovarian cancer, pancreatic cancer, parathyroid cancer, pharyngeal cancer, pituitary adenoma, plasma cell neoplasia, prostate cancer, renal cell carcinoma, retinoblastoma, sarcoma, testicular cancer, thyroid cancer, and uterine cancer.

### A. Pharmaceutical Compositions

The present disclosure further provides compositions comprising populations of cDC. The compositions may comprise pharmaceutically acceptable carriers and diluents, for example saline solution or Hank's Balanced Salt Solution (HBSS). The compositions may comprise pharmaceutically acceptable cryopreservation media, for example CryoStor CS10, which is safe for injection of drug product at acceptable volumes. The compositions may further comprise components that facilitate engraftment. Compositions comprising these populations are useful for cell and tissue replacement and repair, and for generating populations of cDC in vitro and in vivo. The compositions may be formulated as a medicament or delivery device for treating an immune condition.

The cDC of the present disclosure can be supplied in the form of a pharmaceutical composition, comprising an isotonic excipient prepared under sufficiently sterile conditions for human administration. In certain aspects, it may be desirable to disperse the cells using a protease or by gentle mechanical manipulation into a suspension of single cells or smaller clusters. To reduce the risk of cell death upon engraftment, the cells may be treated by heat shock or cultured with about 0.5 U/mL erythropoietin about 24 hours before administration.

For general principles in medicinal formulation, the reader is referred to Cell Therapy: Stem Cell Transplantation, Gene Therapy, and Cellular Immunotherapy, 1996; and Hematopoetic Stem Cell Therapy, 2000. Choice of the cellular excipient and any accompanying elements of the composition will be adapted in accordance with the route and device used for administration.

This disclosure also includes a reagent system, comprising a set or combination of cells that exist at any time during manufacture, distribution, or use. The cell sets comprise any combination of two or more cell populations described in this disclosure, exemplified but not limited to a type of differentiated cell, in combination with undifferentiated pluripotent stem cells or other differentiated cell types, often sharing the same genome. Each cell type in the set may be packaged together, or in separate containers in the same facility, or at different locations, at the same or different times, under control of the same entity or different entities sharing a business relationship.

Pharmaceutical compositions of this disclosure may optionally be packaged in a suitable container with written instructions for a desired purpose.

### B. Therapeutic Uses

The cells provided in certain aspects of this present disclosure can be used for therapy of any subject in need thereof.

To determine the suitability of cell compositions for therapeutic administration, the cells can first be tested in a suitable animal model. At one level, cells are assessed for their ability to survive and maintain their phenotype in vivo. Cell compositions are administered to immunodeficient animals (such as NUDE rats, or animals rendered immunodeficient chemically or by irradiation). Tissues are harvested after a period of engraftment, and assessed as to whether pluripotent stem cell-derived cells are still present.

Other methods to track cells in vivo may be by administering cells that express a detectable label (such as green fluorescent protein, or β-galactosidase); that have been prelabeled (for example, with BrdU or [³H]thymidine), or by subsequent detection of a constitutive cell marker (for example, using human-specific antibody). The presence and phenotype of the administered cells can be assessed by immunohistochemistry or ELISA using human-specific antibody, or by RT-PCR analysis using primers and hybridization conditions that cause amplification to be specific for human polynucleotides, according to published sequence data.

After adequate testing, differentiated cells of this disclosure can be used for tissue reconstitution or regeneration in a human patient or other subject in need of such treatment. The cells are administered in a manner that permits them to graft or migrate to the intended tissue site and reconstitute or regenerate the functionally deficient area.

With respect to the therapeutic methods of the present disclosure, it is not intended that the administration of cDC to a mammal be limited to a particular mode of administration, dosage, or frequency of dosing; the present disclosure contemplates all modes of administration, including intramuscular, intravenous, intrarticular, intralesional, subcutaneous, or any other route sufficient to provide a dose adequate to prevent or treat a disease. The cDC may be administered to the mammal in a single dose or multiple doses. When multiple doses are administered, the doses may be separated from one another by, for example, one week, one month, one year, or ten years. One or more growth factors, hormones, interleukins, cytokines, small molecules or other cells may also be administered before, during, or after administration of the cells to further bias them towards a particular cell type.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Production of Dendritic Cells from iPSCs

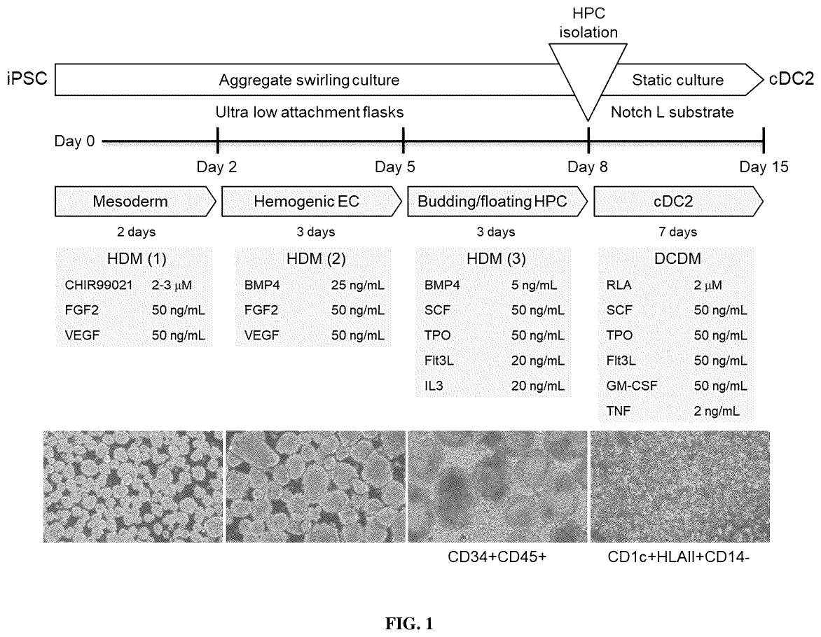

iPSCs were maintained on vitronectin-coated plates in E8 medium and were dissociated at 50% confluence after 10 min incubation in 0.5 mM EDTA/PBS, counted and plated into ultra-low attachment flasks (Corning) at 5×10⁴/mL in HDM1 supplemented with 1 μM H1152 ROCK inhibitor (day 0). Flasks were placed on the orbital shaker set on 25-30 rpm for uniform size aggregate formation and following culture agitation throughout HPC differentiation. 80% medium was changed every day. Cultures were processed through (1) mesoderm (d1-2), (2) hemogenic endothelial cell (EC, days 3-5) and (3) budding/floating hematopoietic progenitor cell (HPC, days 6-8) stages in HDM (see composition in Table 1, FIG. 1) variants supplemented with indicated stage-specific inductive factors, designated as HDM1, HDM2 and HDM3, respectively.

On day 8, CD34⁺CD43⁺CD45^{+/-} HPCs were collected from a 30 μM filtrate of the entire culture by centrifugation, counted and plated on the Notch ligand (L)-coated plates (DLL1-Fc, 0.25 μg/cm²; DLL4-Fc, 0.25 μg/cm²; JAG1-Fc, 1 μg/cm²; or JAG2-Fc, 0.5 μg/cm² in the 0.1-1 μg/cm² range) at 5×10³/cm² density for 1-week of cDC2 differentiation in DCDM (see composition in Table 2) supplemented with Vitamin A as retinyl acetate (RLA) and indicated DC supportive cytokines SCF (50 ng/mL), TPO (50 ng/mL), FLT3L (50 ng/mL), GM-CSF (50 ng/mL), and TNF (2 ng/mL) (FIG. 1). cDC2 emerged as a homogeneous (e.g., more than 90%) population of CD1c+CD14−HLA class II+ cells on days 6-7. For harvesting, cDC2 were suspended and collected by gentle pipetting. The remaining attached cDC2 were collected after 10 min incubation in 0.5 mM EDTA/PBS.

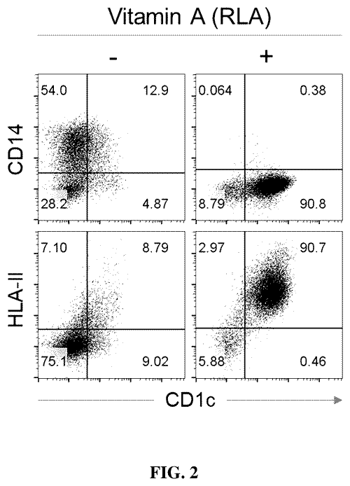

To determine the effect of Vitamin A on the DC differentiation, HPCs were cultured 7 days in DCDM supplemented with DC supportive cytokines SCF, TPO, FLT3L, GM-CSF, and TNF without (control) or with Vitamin A in form of retinyl acetate (RLA). Vitamin A was added at a dose normally detected in human serum (2 μM). While mostly CD14+ monocytic cells were detected in the control cultures, adding Vitamin A resulted in a near complete suppression of CD14+ cells and development of a more than 90% pure CD1c+CD14− population (FIG. 2). In contrast to CD14+ cells, the emerging CD1c+CD14− cells instantly express HLA class II (HLA-II) at high level without activation—a feature of cDCs. These data highlight distinct differentiation pathways for monocytic and cDC lineages and identify Vitamin A as the determining factor of the cDC2 lineage.

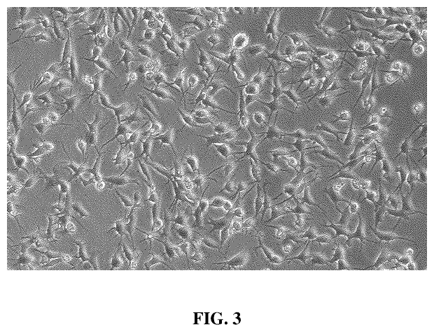

Next, the morphology of the dendritic cells was evaluated after 6 days of differentiation on the RETRONECTIN®-coated plates. It was observed that the CD1c+CD14− cells emerged as a morphologically homogenous population of small semi-adherent cells with well-developed multiple dendrites (FIG. 3).

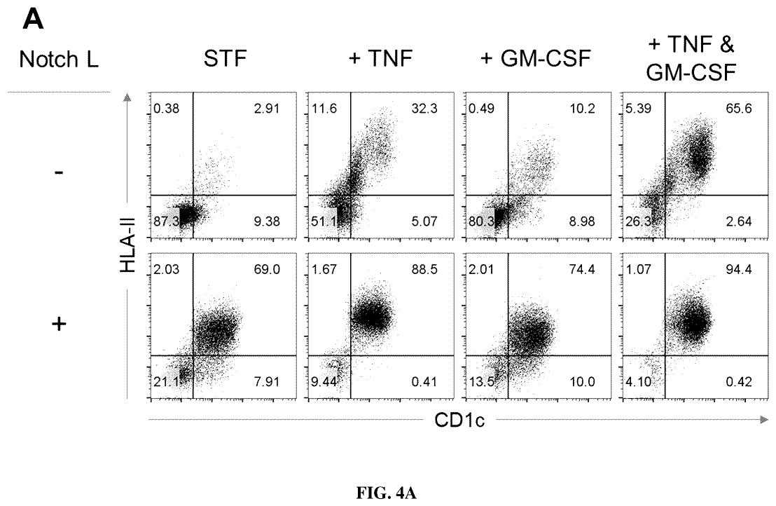

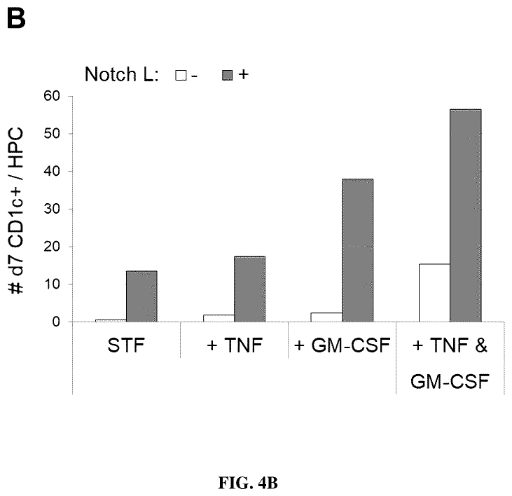

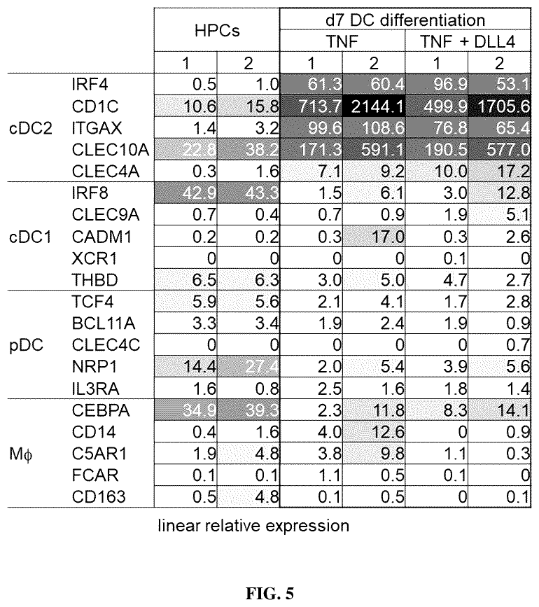

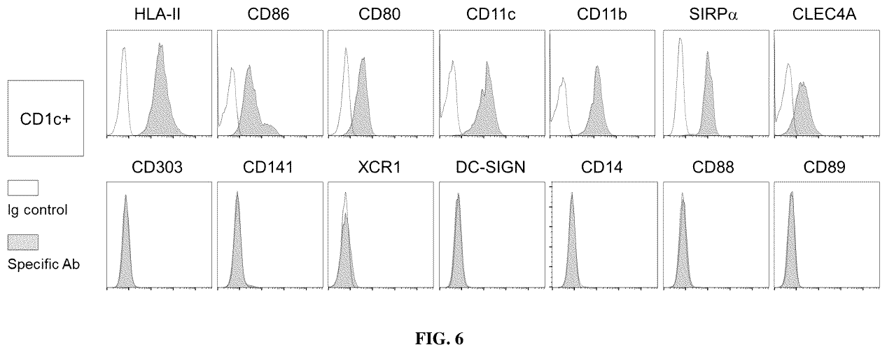

It was further found that Notch signaling strongly promotes Vitamin A-induced cDC2 differentiation. HPCs were cultured 7 days on plates coated (+) or not (−) with Notch ligand (Notch L: DLL4-Fc, 0.25 μg/cm²) in DCDM supplemented with Vitamin A (retinyl acetate at 2 μM) and HPC growth factors only (STF: SCF, TPO, FLT3L) or TNF and GM-CSF added to STF either alone or in combination (TNF & GM-CSF). cDC2 (CD1c+HLA class II+) phenotyping (FIG. 4A) showed that both TNF and GM-CSF were needed to induce efficient cDC2 differentiation without Notch activation, whereas only HPC growth factors (STF) were sufficient to generate highly enriched cDC2 in Notch L+ conditions. Thus, Notch activation replaces the need for TNF and GM-CSF to induce cDC2 in the presence of Vitamin A. Nevertheless, both TNF and GM-CSF positively contributed to cDC2 development in Notch L+ conditions as well. The quantitative cDC2 yields (FIG. 4B) showed that GM-CSF greatly promoted, and TNF additionally contributed to cDC2 growth in Notch L+ cultures. Moreover, TNF significantly up-regulated HLA class II expression by cDC2 (FIG. 4A). The resulting conditions for the most efficient cDC2 generation included all tested components: Vitamin A, Notch L, and DC cytokines. With this procedure, more than 90% pure cDC2 could be generated with yields of more than 50 per HPC. Considering >50 HPC yields from iPSC, overall efficiency of cDC2 production from iPSC is about 2500 per iPSC in 15 days. It is important to note that in absence of Vitamin A, no substantial cDC2 production was detected in any conditions, with or without Notch activation. The cell percentage data presented is summarized in Table 3.

TABLE 3
Percentages of cell subpopulations under each condition listed
in FIG. 2. The first column shows if the cell expresses the
marker listed (CD14, CD1c and/or HLA class II (HLA-II), and
whether there is positive expression (+) or the absence
of positive expression (−). Additionally, the first column
indicates if the cells are cultured in the presence (+) or
absence (−) of retinyl acetate (RLA).

| | |
|---|---|
| CD14+ CD1c− | 54.0 |
| RLA− | |
| CD14+ CD1c+ | 12.9 |
| RLA− | |
| CD14− CD1c− | 28.2 |
| RLA− | |
| CD14− CD1c+ | 4.87 |
| RLA− | |
| CD14+ CD1c− | 0.064 |
| RLA+ | |
| CD14+ CD1c+ | 0.38 |
| RLA+ | |
| CD14− CD1c− | 8.79 |
| RLA+ | |
| CD14− CD1c+ | 90.8 |
| RLA+ | |
| HLA-II+ CD1c− | 7.10 |
| RLA− | |
| HLA-II+ | 8.79 |
| CD1c+ RLA− | |
| HLA-II− CD1c− | 75.1 |
| RLA− | |
| HLA-II− CD1c+ | 9.02 |
| RLA− | |
| HLA-II+ CD1c− | 2.97 |
| RLA+ | |
| HLA-II+ | 90.7 |
| CD1c+ RLA+ | |
| HLA-II− CD1c− | 5.88 |
| RLA+ | |
| HLA-II− CD1c+ | 0.46 |
| RLA+ | |

TABLE 4
Percentages of each subpopulation of cells displaying the combination
of cell markers in the first column (HLA class II and CD1c), from
FIG. 3. Additionally, the first column indicates if Notch L was
added (Notch L+) or not (Notch L−). The top row indicates
the growth factors added (STF, or TNF, or TNF and GM-CSF).

| | | | | TNF AND |
|---|---|---|---|---|
| | STF | TNF | GM-CSF | GM-CSF |
| HLA II+ | 0.38 | 11.6 | 0.49 | 5.39 |
| CD1c− | | | | |
| Notch L− | | | | |
| HLA II− | 87.3 | 51.1 | 80.3 | 26.3 |
| CD1c− | | | | |
| Notch L− | | | | |
| HLA II+ | 2.91 | 32.3 | 10.2 | 65.6 |
| CD1c+ | | | | |
| Notch L− | | | | |
| HLA II− | 9.38 | 5.07 | 8.98 | 2.64 |
| CD1c+ | | | | |
| Notch L− | | | | |
| HLA II+ | 2.03 | 1.67 | 2.01 | 1.07 |
| CD1c− | | | | |
| Notch L+ | | | | |
| HLA II− | 21.1 | 9.44 | 13.5 | 4.10 |
| CD1c− | | | | |
| Notch L+ | | | | |
| HLA II+ | 69.0 | 88.5 | 74.4 | 94.4 |
| CD1c+ | | | | |
| Notch L+ | | | | |
| HLA II− | 7.91 | 0.41 | 10.0 | 0.42 |
| CD1c+ | | | | |
| Notch L+ | | | | |

mRNA profiling confirmed the highly selective cDC2 differentiation. HPCs from two iPSC lines were differentiated 7 days on plates coated or not with Notch ligand DLL4-Fc (0.125 μg/cm²) in DCDM supplemented with Vitamin A, SCF, TPO, FLT3L, GM-CSF and TNF cytokines, referred to as “TNF” and “TNF+DLL4” conditions for 7 day DC differentiation. Total RNA was extracted and analyzed by Illumina RNA sequencing. Selected markers representative of specific DC and macrophage lineages (e.g., cDC2 markers IRF4, CD1C, ITGAX, CLEC10A, and CLEC4A) revealed the highly selective differentiation to cDC2 lineage in both TNF and TNF+DLL4 conditions. No inducible and/or consistent expression of cDC1 (e.g., IRF8, CLEC9A, CADM1, XCR1, and THBD), plasmacytoid DC (pDC) (e.g., TCF4, BCL11A, CLECL4C, NRP1, and IL3RA) or macrophage (Mφ) (e.g., CEBPA, CD14, C5AR1, FCAR, and CD163) markers further confirmed that the differentiated CD1c+CD14− cells most closely resemble represent cDC2-type dendritic cells.

Next, it was shown that CD1c+ cell phenotyping confirmed cDC2 identity. Although CD1c is defining marker of human cDC2 lineage in absence of monocytic markers (CD14), iPSC derived CD1c+ cells were tested for expression of additional DC type defining markers. CD1c+ cells express common DC (CD11c) and APC (HLA class II, CD80, CD86) markers along with markers associated with cDC2 lineage (CD172a/SIRPα, CLEC4A, CD11b) but not plasmacytoid DC (CD303), cDC1 (CD141, XCR1), monocyte-derived DC (CD209/DC-SIGN) or monocytes/macrophages (CD14, CD88, CD89). Thus, the cDC2 identity of the iPSC-derived CD1c+ cells generated by present methods was confirmed.

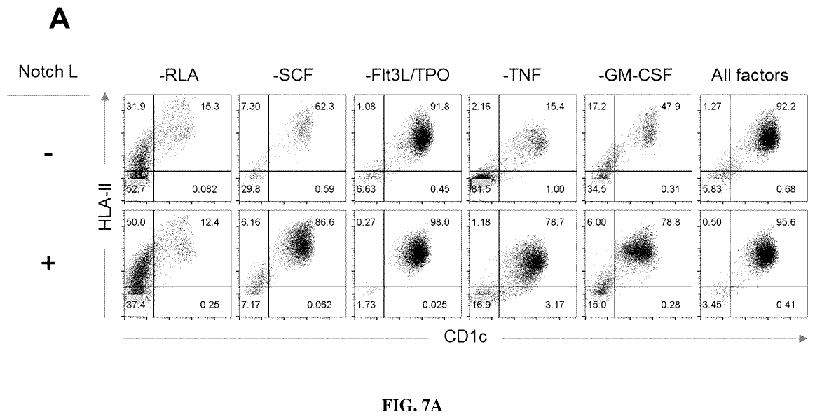

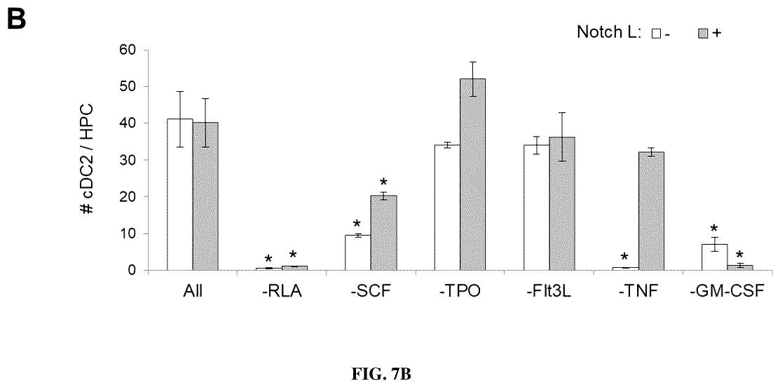

It was also shown that iPSC-derived cDC2 acquired a mature phenotype after short-term exposure to pathogen-associated or pro-inflammatory factors. cDC2 generated in a 6 day differentiation culture were collected, washed, and replated at 2.5×10⁵/mL density in DCDM supplemented with SCF, FLT3L and GM-CSF at 50 ng/mL each either alone (no stimulus control) or with added activators: (1) DC-activating pro-inflammatory cocktail (50 ng/mL TNF, 50 ng/mL IL1A, 10 ng/mL IL6, and 1 μM PGE2); (2) 25 μg/mL particulate zymosan (yeast β-glucan/protein complexes, PAMP ligand for TLR2/Dectin-1 PRR). After 24-hour incubation, expression of DC maturation/activation marker CD83, HLA class II, and co-stimulatory (CD80, CD86, CD40) molecules was determined. While in control cultures cDC2 remain quiescent (CD83−), they become CD83+ and strongly up-regulate expression of HLA class II and co-stimulatory CD80, CD86, CCR7 and CD40 molecules in both activation cultures. Thus, cDC2 generated by the present methods were functionally competent to quickly respond and acquire a mature phenotype after exposure to DC-activating factors. Histogram gates in FIG. 7 show % positive cells relative to isotype Ig control. Values in italic show mean fluorescence intensity (MFI) relative to isotype Ig control (specific Ab MFI/isotype Ig control MFI).

cDC2 differentiation was tested in conditions with individual inductive factor withdrawal in both Notch L− and Notch L+ (0.25 μg/cm² DLL4-Fc) cultures (FIG. 7). Vitamin A (retinyl acetate, RLA), SCF and GM-CSF were needed for efficient cDC2 differentiation in both Notch L− and Notch L+ cultures. TNF was only needed for Notch L− cultures, whereas FLT3L/TPO were found to be optional.

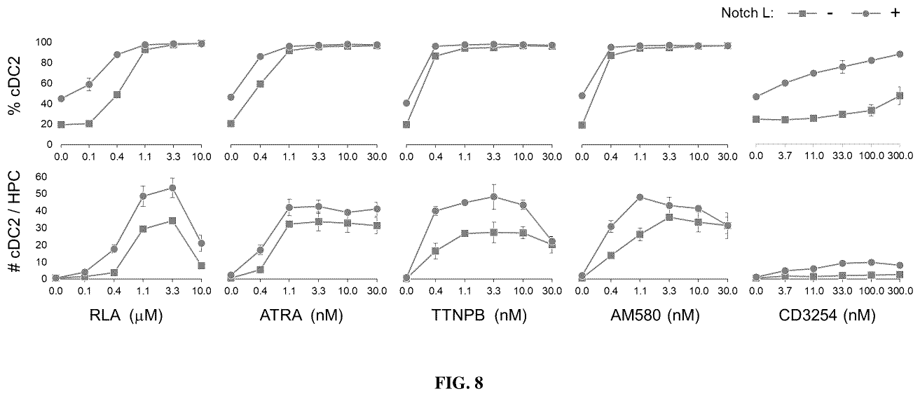

Testing different retinoids revealed a key role of RARα signaling in cDC2 differentiation (FIG. 8). cDC2 differentiation was tested in the presence of different agonists of the retinoid signaling pathway in both Notch L− and Notch L+ (DLL4-Fc, 0.125 μg/cm²) cultures. The cDC2-inductive effect of Vitamin A (e.g., retinyl acetate, RLA) could be reproduced by natural (e.g., all-trans retinoic acid, ATRA), synthetic (e.g., TTNPB) pan-RAR and RARα selective (e.g., AM580) agonists, but not RXR-specific CD3254. This indicates that Vitamin A-mediated cDC2 differentiation can be attributed to RAR (specifically RARα) signaling. Notably, Vitamin A is effective and optimal at physiological doses in the 1-3 μM range and toxic at higher concentrations, while tested pan-RAR and RARα agonists were effective at 1000× lower concentrations and had wider optimal ranges (e.g., 1-10+nM).

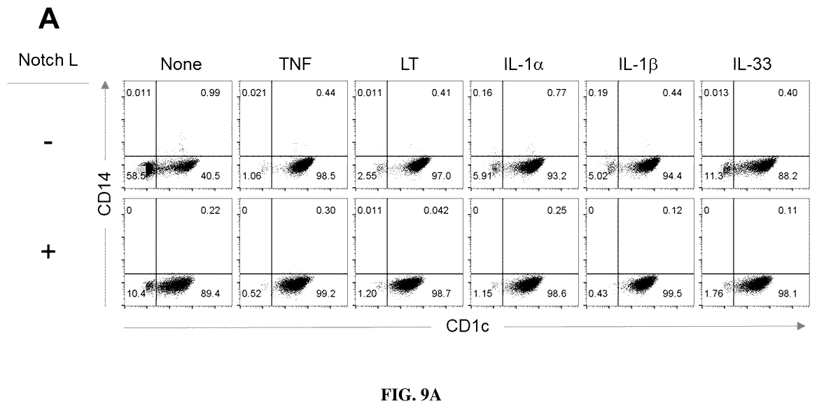

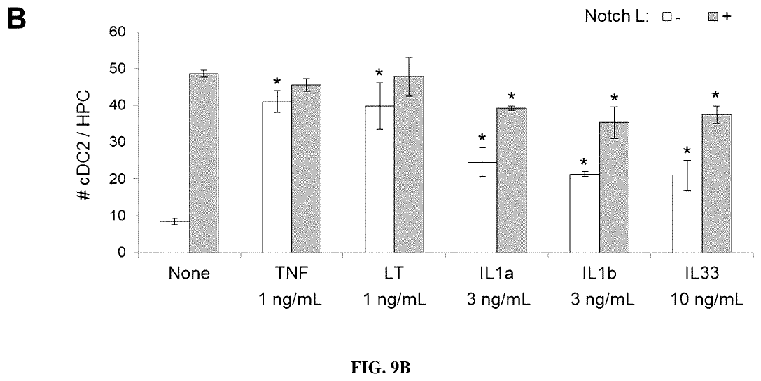

TNF was found to be important for cDC2 differentiation in cultures without Notch activation (FIG. 9). Another functionally similar pro-inflammatory cytokine IL-1β (5 ng/mL) was tested and found less efficient than TNF (2 ng/mL) in terms of cDC2 purity and yields. However, both factors support cDC2 differentiation in Notch L− cultures, and IL-1β can be used to replace TNF.

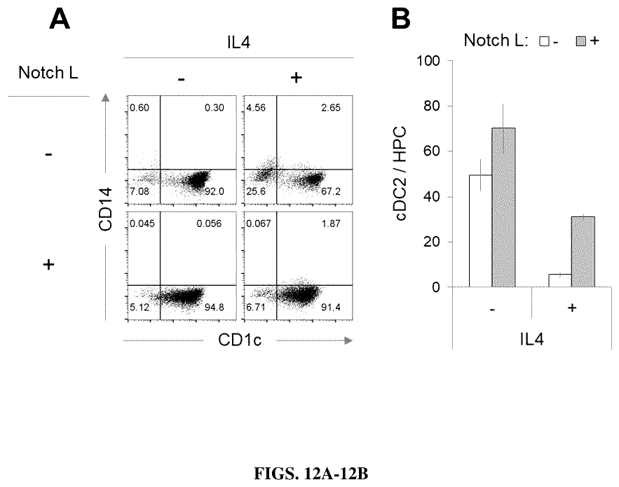

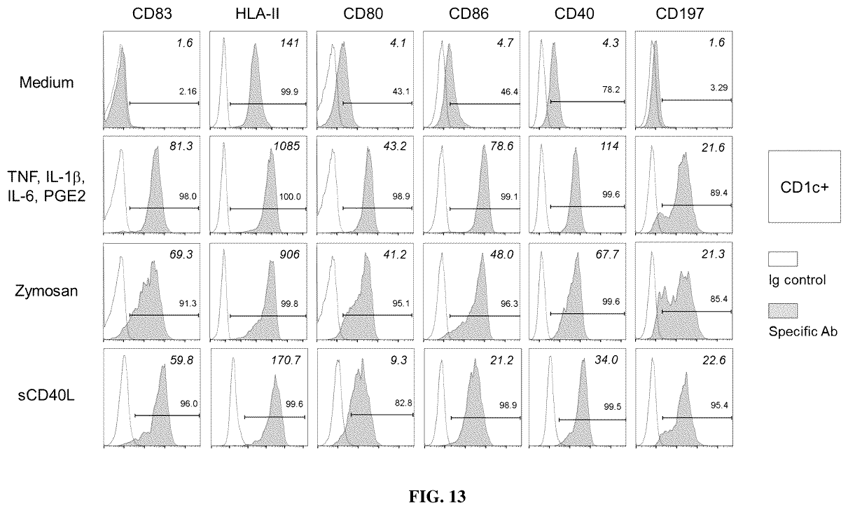

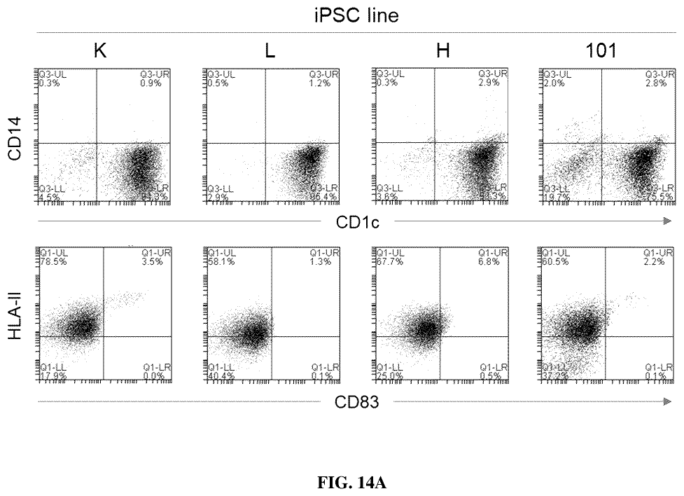

IL4 is commonly used to generate DCs from primary monocytes in combination with GM-CSF. It is also included in the most published methods for DC generation from iPSCs. However, the present studies found IL-4 was strongly suppressive for Vitamin A-induced cDC2 differentiation, especially in Notch L− cultures (FIG. 12).

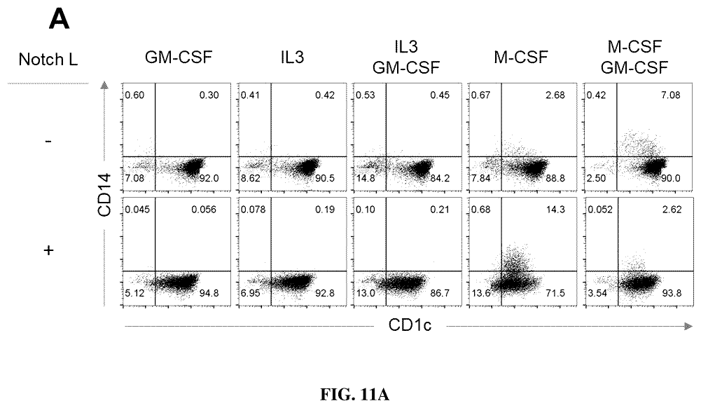

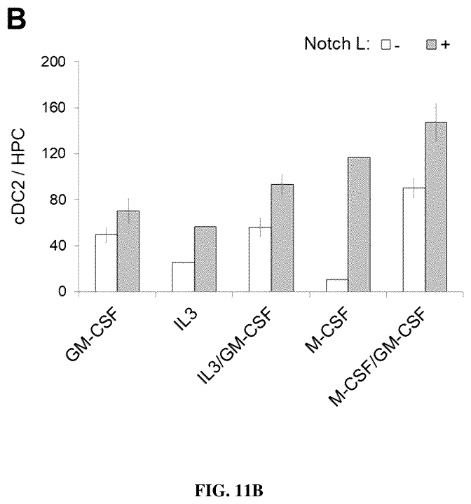

GM-CSF is a commonly used factor to promote DC growth and differentiation. IL-3 is reported as an alternative factor, though less efficient, but M-CSF is commonly used for monocyte/macrophage growth and differentiation, not DCs. It is remarkable that under cDC2 differentiation conditions with Vitamin A, both IL-3 and M-CSF support cDC2 growth and differentiation. IL-3 alone is less efficient than GM-CSF (as previously reported) but may improve cDC2 generation when combined with GM-CSF. M-CSF alone supports highly efficient cDC2 differentiation only in Notch L+ conditions, but in combination with GM-CSF, results in about 2-times higher efficiency of cDC2 generation in both Notch L− and Notch L+ cultures (FIG. 11). These data show that GM-CSF, IL-3 and M-CSF can be used to support cDC2 growth and differentiation, and their combinations provide additional advantages to improve efficiency.

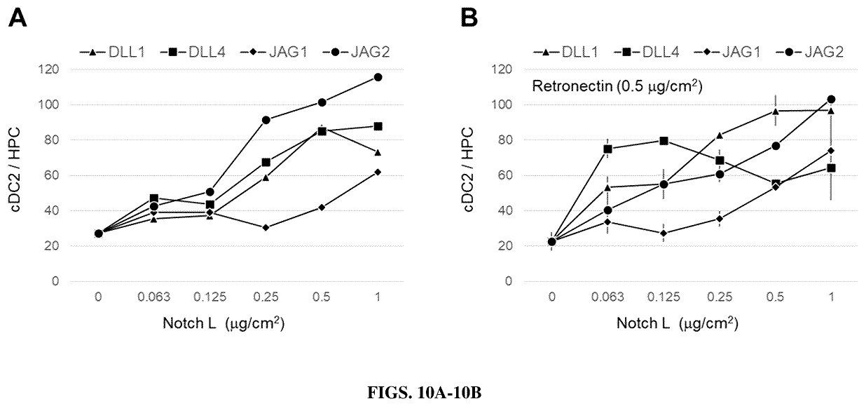

All data for Notch L+ cultures were obtained with DLL4 Notch ligand. Other available recombinant Notch L-Fc chimera proteins (DLL1, JAG1 and JAG2) were tested and found supportive for cDC2 differentiation as well. As expected, different Notch ligands show different efficiency, which also depends on their absorption to plastic with or without RETRONECTIN®. In cultures without RETRONECTIN® (FIG. 10A), cDC2 supportive activity of Notch ligands was distributed in the following order: JAG2>DLL1=DLL4>>JAG1, while in cultures with RETRONECTIN® (FIG. 10B), activity of DLL1 and especially DLL4 was significantly increased and resulted in the different hierarchy: DLL4>DLL1>JAG2>>JAG1. Unexpectedly, JAG2 was found superior in conditions without RETRONECTIN®. DLL1 and DLL4 have similar efficiency, albeit much stronger DLL4 activity in presence of RETRONECTIN®. JAG1 activity was relatively weak in all conditions. Nevertheless, all tested Notch ligands can be used to support cDC2 differentiation.

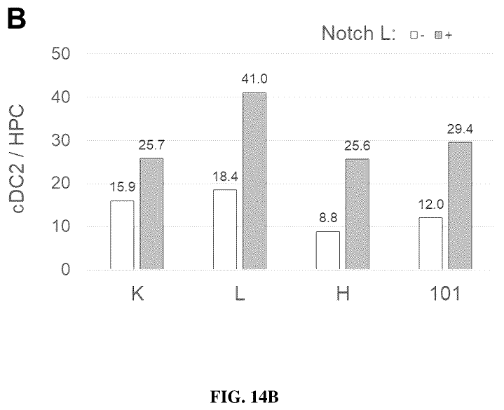

It was further shown that cDC2 cells can be efficiently differentiated from iPSC reprogrammed from PBMC other than T cells (FIG. 14). Much of the data shown included cDC2 differentiated from iPSC lines derived from T cells (“TiPSC” lines). To confirm that cDC2 could be derived from standard iPSC lines, the cDC2 differentiation protocol was performed on four iPSC lines that had been reprogrammed from CD34+ PBMC (Lines K, L and H) or expanded PBMC (Line 101) that were not T cells or B cells (“non-TiPSC”, confirmed by sequencing of the non-rearranged TCR and BCR loci of the iPSC lines). Flow cytometry demonstrated that all lines efficiently differentiated to cDC2 dendritic cells (CD1c+CD14−HLA Class II+). CD83 is not or barely expressed, thus confirming immature non-activated cDC2 differentiation from various iPSC lines.

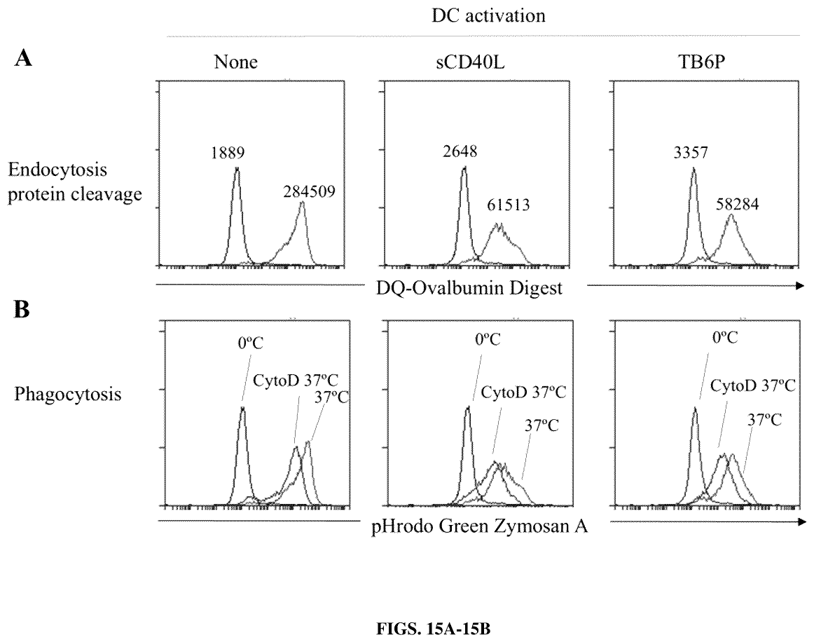

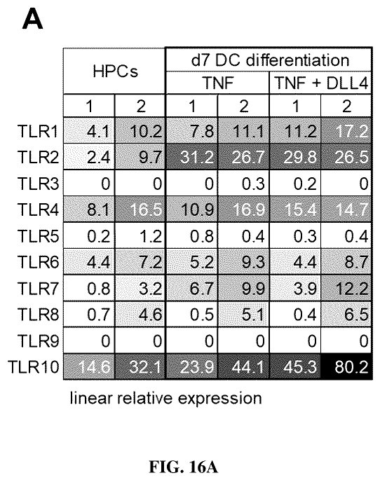

cDC2 phagocytosis and protein processing function by cDC2 cells is shown in FIG. 15. cDC2 cells were differentiated from iPSC-derived HPCs for 7 days using vitamin A and TNF on a DLL4-Fc-coated surface, and then either not activated (“None”) or activated with soluble CD40 ligand (“sCD40L”) or TNF/IL1b/IL-6/Prostaglandin E2 (“TB6P”) for 24 hours. (FIG. 15A) The endocytic activity was assessed by measuring the uptake of the fluorescent reporter DQ-ovalbumin (receptor-mediated endocytosis). iPSC-derived cDC2 cells were incubated with 10 μg/10⁵ cells DQ-Ovalbumin at 0° C. or 37° C. for 15 minutes. The fluorescence of ovalbumin labeled with BODIPY FL dye (DQ-Ovalbumin) is self-quenched until the ovalbumin is taken up via the mannose receptor and degraded by endolysosomal proteases. Flow histogram gates showed % positive cells at 37° C. relative to 0° C. The mean fluorescence intensities (MFI) for the different fluorescent cell populations are indicated in each histogram. Non activated cDC2 showed the strongest receptor mediated endocytosis. (FIG. 15B) To measure phagocytosis, the cDC2 cells were incubated with 10 μg/ml pHrodo Green Zymosan A Bioparticles Conjugate that is fluorescent in the low pH environment of lysosomes and endosomes. As expected, phagocytosis is greatly reduced if the 1-hour incubation is carried out at 0° C., or if the cells are pretreated with the specific inhibitor cytochalasin D (Cyto D, 10 μM) for 15 minutes prior to addition of the Bioparticles. Consistent with the literature on primary dendritic cells, non-activated (“immature”) cDC2 showed the strongest phagocytosis.

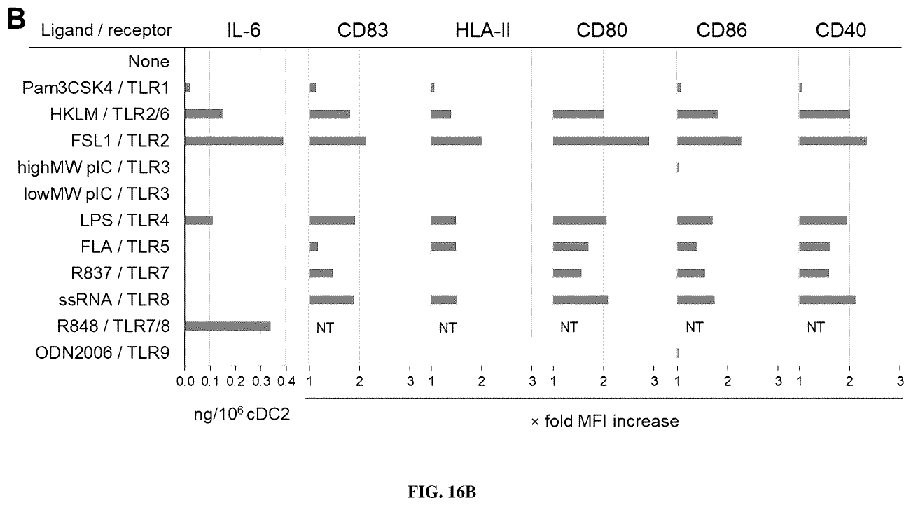

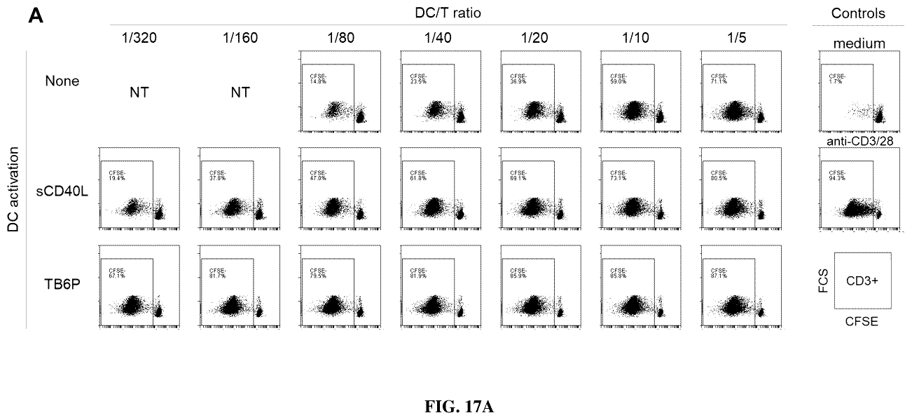

Further, cDC2-specific TLR expression and functional response were shown (FIG. 16). Different DC types can be distinguished by characteristic expression of toll-like receptors (TLR). While cDC1 and pDC express TLR3 and TLR9 in a restricted manner, cDC2 cells express a wide range of other TLRs with highest expression of TLR2. (FIG. 16A) mRNA sequencing of cells differentiated from two iPSC lines (line 1 or line 2) demonstrated low level TLR gene expression in HPCs (TLR1,2,4,6-8,10), which was maintained or up-regulated (TLR2,7,10) in the differentiated cDC2 cells. (FIG. 16B) When cDC2 cells were treated with a panel of TLR-specific ligands (InvivoGen), cDC2 cells responded to TLR2,4 and 7/8 ligands as measured by IL-6 production and up-regulation of activation markers (CD83, HLA-II, CD80, CD86, CD40) and did not have a detectable response to TLR3,9 ligands. This TLR responsiveness profile is characteristic of cDC2 cells. IL-6 was determined by Luminex assay (R&D Systems) and expressed as ng/10⁶ cDC2 cells produced in a 32-hour culture. Expression of activation markers was measured by flow cytometry mean fluorescence intensity (MFI) compared to non-activated control (MFI in activated culture/MFI in non-activated control). NT—not tested.

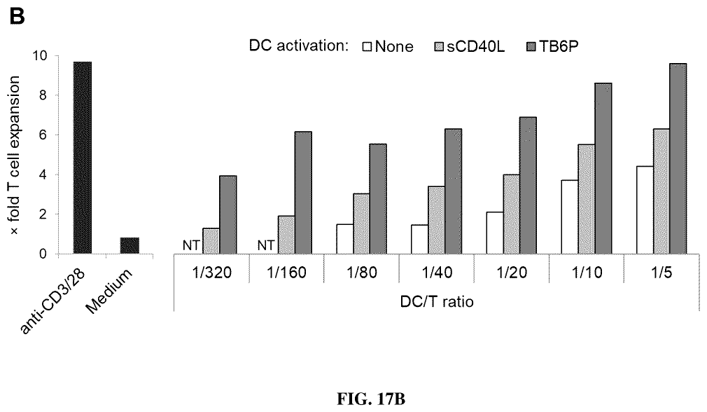

iPSC-derived cDC2 dendritic cells supported the allogenic proliferation of primary T cells. cDC2 cells were differentiated from iPSC-derived HPCs for 7 days using the conditions with vitamin A and TNF on DLL4-Fc-coated surface, and then either not activated (“None”) or activated with soluble CD40 Ligand (“sCD40L”) or TNF/IL1b/IL-6/Prostaglandin E2 (“TB6P”) for 24 hours. The cDC2 cells were washed three times prior to co-culture to remove activating compounds. Allogenic primary T cells were isolated from PBMCs using the Dynabeads Untouched Human T cell Kit to >90% purity (2.4% CD19+, <1% CD14+, <1% CD56+). The T cells were labeled with CellTrace CFSE to track proliferation. 105 T cells/well were co-cultured with decreasing ratios of allogenic cDC2 cells for 1 week. (FIG. 17A) No significant T cell proliferation is observed when allogenic cDC2 cells are not added (“medium”), while strong proliferation is triggered with anti-CD3/CD28 antibodies (“anti-CD3/28”), as seen by the diluted CFSE fluorescent intensity in divided T cells. Co-culture with allogenic cDC2 cells triggers robust proliferation of T cells (both CD4+ and CD8+ T cells). The response is particularly potent using TB6P-activated cDC2 cells, where high levels of proliferation are seen at a DC/T cell ratio as low as 1:320 (approximately 300 cDC2 cells per well). (FIG. 17B) Enumeration of the T cells after 1 week of co-culture quantifies the level of expansion. The most potent T cell stimulation is achieved using TB6P-activated cDC2 cells, which trigger a 4-fold expansion at a 1:320 ratio, and up to 10-fold expansion at higher ratios.

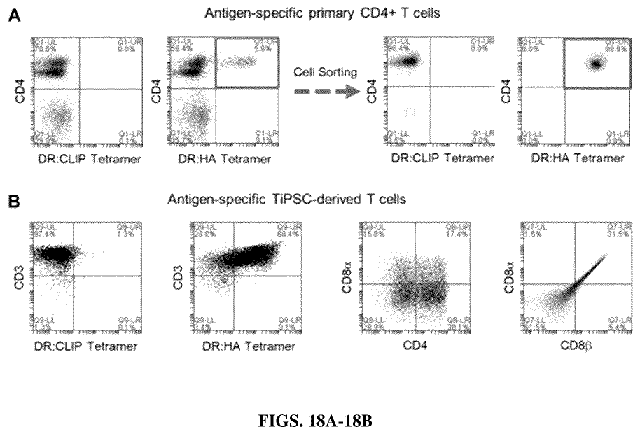

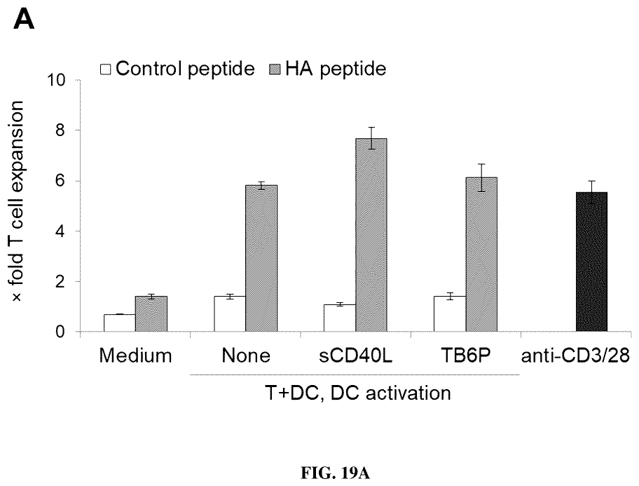

Antigen-specific TiPSC-derived T cells were derived that recognize Influenza HA (306-318) peptide in the context of HLA DRB1*01:01. (FIG. 18A) PBMC from an HLA DRB1*01:01+ donor were repeatedly stimulated with Influenza HA (306-318) peptide and autologous feeder cells until a population of peptide-specific CD4+ T cells was detectable using a fluorophore-labeled HLA DRB1*01:01/HA (306-318) tetramer reagent (“DR:HA Tetramer”). No staining is detected using a DRB1*01:01 tetramer bearing an irrelevant control peptide (DR:CLIP Tetramer). The DR:HA Tetramer+/CD4+ population was sorted using FACS, resulting in a >99% population of antigen-specific CD4+ T cells. (FIG. 18B) The antigen-specific T cells were reprogrammed to T cell-derived iPSC clones (“TiPSC”) using a Sendai virus-based reprogramming kit. TiPSC clones were screened for those with TCR rearrangements specific for the HA antigen by differentiating the TiPSC clones to T cells and staining using the DR:HA Tetramer reagent. T cells made from a clone (Line 2) were differentiated for 21 days past the HPC stage and used for cDC2 co-culture experiments. T cells made from this clone express a TCR specific for DRB1*01:01/HA (306-318) as demonstrated by DR:HA Tetramer binding. Expression of CD4 and CD8 on these cells is heterogeneous, with approximately 38% CD4+CD8−, 16% CD8+CD4−, 17% CD4+CD8+, and 29% CD4−CD8−. All CD8+ cells express the CD8α/β heterodimer.

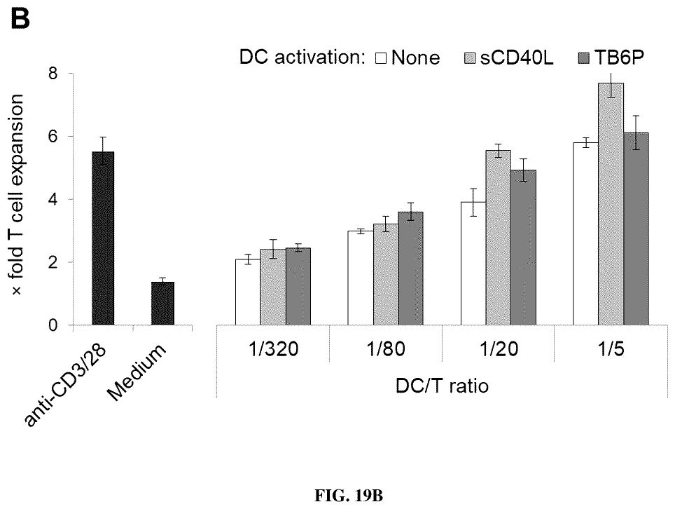

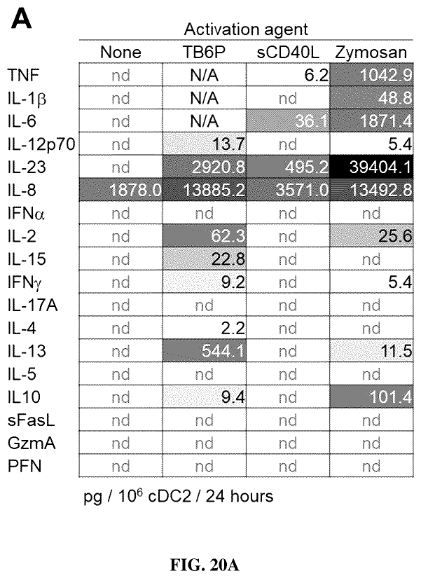

iPSC-derived cDC2 dendritic cells were shown to present HLA Class II peptide for antigen-specific stimulation of iPSC-derived T cells. cDC2 cells were differentiated from iPSC-derived HPC for 7 days using vitamin A and TNF on a DLL4-Fc-coated surface, and then either not activated (“None”) or activated with soluble CD40 Ligand (“sCD40L”) or TNF/IL1b/IL-6/Prostaglandin E2 (“TB6P”) for 24 h. The cDC2 cells were washed three times prior to co-culture to remove activating compounds and pre-incubated with influenza HA (306-318) peptide (“HA peptide”, 1 μg/ml) or irrelevant CLIP peptide (“Control peptide”, 1 μg/ml). Autologous iPSC-derived T cells specific for Influenza HA (306-318) peptide in the context of HLA DRB1*01:01 were differentiated from the TiPSC line. 2×10⁵ iT cells/well were co-cultured with different ratios of autologous, peptide-loaded cDC2 cells for 1 week. (FIG. 19A) No significant T cell proliferation was observed when cDC2 cells are not added (“medium”), while strong proliferation is triggered with anti-CD3/CD28 antibodies (anti-CD3/28”), as seen by enumeration of the T cells after 1 week of co-culture. Co-culture with autologous cDC2 cells (1:5 DC/T ratio) triggers robust proliferation of iT cells, but only in the presence of HA peptide, demonstrating a Class II-restricted antigen-specific response. Similar iT cell proliferation is triggered by activated or non-activated cDC2 cells. (FIG. 19B) Titration of the DC:T ratio demonstrates reduced antigen-specific T cell proliferation at lower DC:T ratios, but still detectable at 1:320. Unlike what was observed using primary T cells, activated cDC2 cells are not significantly more potent than non-activated cDC2 cells at stimulating iT cells, although slightly better responses were observed using CD40L-activated cDC2 cells. This may be due to the iPSC-derived T cells having an immature phenotype and lacking many of the co-receptors expressed by primary naïve T cells.

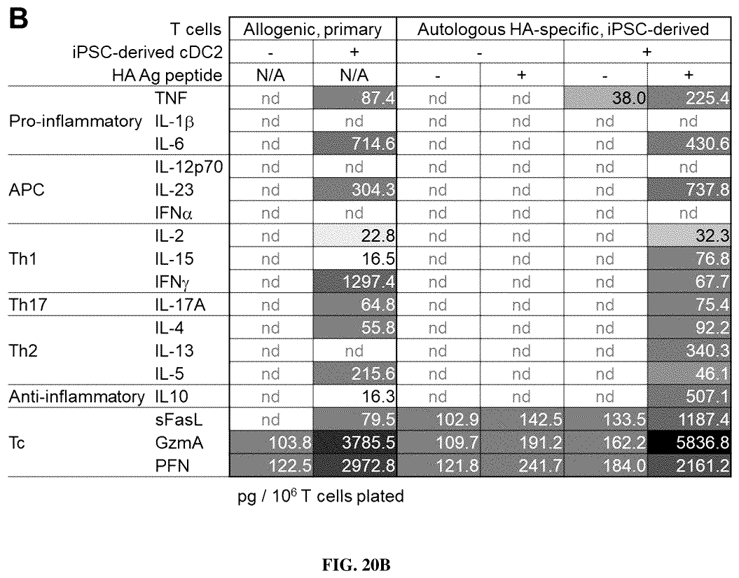

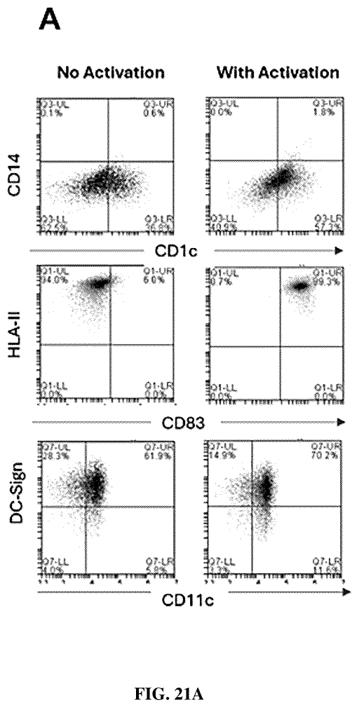

Further studies showed cDC2-specific cytokine production and T cell activation response. (FIG. 20A) Different DC types can be distinguished by characteristic cytokine secretion profile in response to activation stimuli. While cDC1 and pDC produce IL-12p70 and type I IFNα/β, respectively, cDC2 mostly produce pro-inflammatory IL-23, IL-6 and TNF. cDC2 cells generated in 7-day differentiation culture were plated at 2.5×10⁵/mL density in DCDM supplemented with SCF, FLT3L and GM-CSF at 50 ng/mL each either alone (“none”) or with added activators: (1) DC-activating pro-inflammatory cocktail “TB6P” (50 ng/mL TNF, 10 ng/mL IL10, 10 ng/mL IL6, and 1 μM PGE2); (2) 50 ng/mL soluble CD40 ligand (“sCD40L”); and (3) 10 μg/mL particulate zymosan A (yeast β-glucan/protein complexes, PAMP ligand for TLR2/Dectin-1). Culture supernatants were collected after 24-hours. Testing cytokines in supernatants from activated cDC2 cultures revealed a predominant production of IL-23, IL-6 and TNF, but not IL-12p70 or IFNα, thus confirming cDC2-specific cytokine secretion. (FIG. 20B) When cDC2 cells were co-cultured with allogenic peripheral blood CD3+ T cells or autologous influenza hemagglutinin (HA)-specific HLA DRB1-restricted iPSC-derived T cells, the characteristic production of cDC2-specific IL-23, IL-6, TNF was observed. In addition, cytokines associated with Th1 (IFNγ), Th17 (IL-17A), Th2 (IL-4) and cytotoxic T cell (GzmA) responses were detected, thus indicating a profound multifunctional T cell stimulatory activity by cDC2. Specific response in cDC2 co-cultures with autologous HA-specific T cells only in presence of HA antigen peptide also confirms the HLA class II-restricted APC function in cDC2. To avoid interference with the cytokine assays, IL-2 was not added to the co-culture media. Cytokines were measured by multiplex Luminex and LEGENDplex assays (R&D Systems, BioLegend) and expressed as pg per 10⁶ cDC2 in 24 hours (A) or pg per 10⁶ T cells plated (B). cDC2 were added to T cells at 1/5 ratio. N/A—not applicable, nd—not detectable (below assay detection limit).

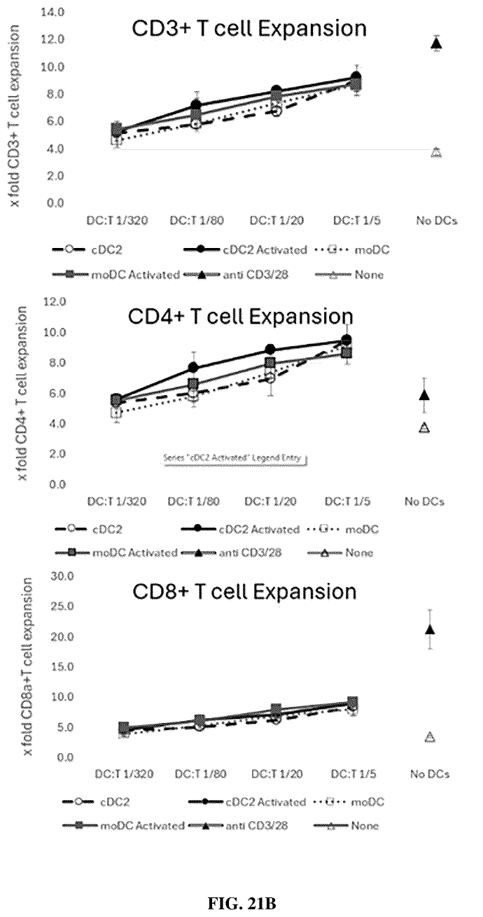

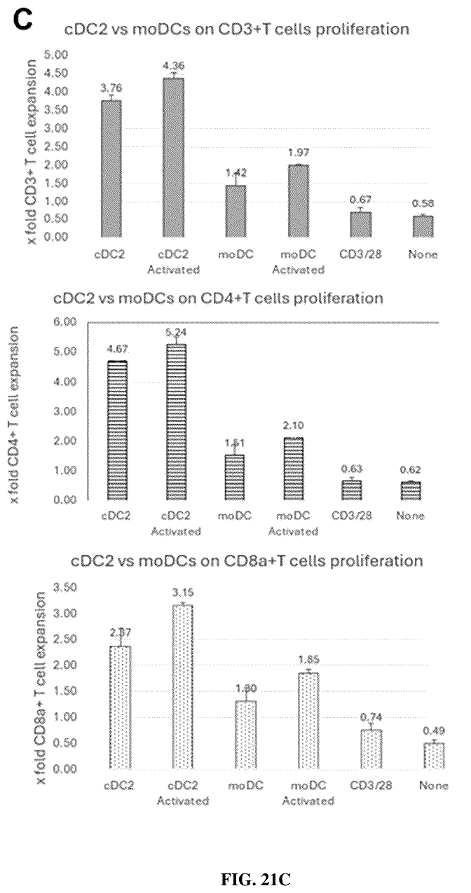

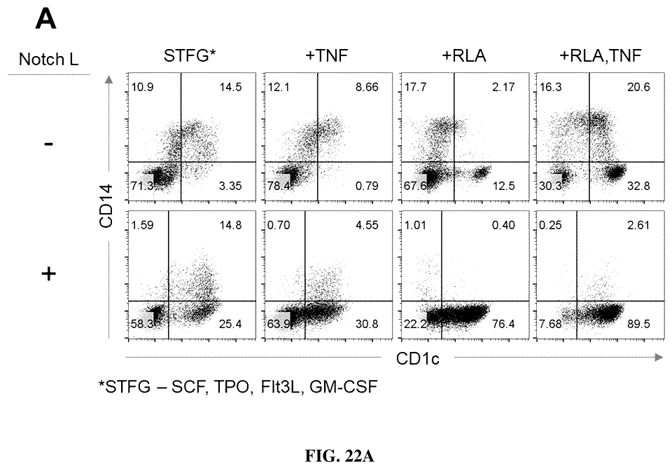

T cells stimulated by iPSC-derived cDC2 cells were compared to primary dendritic cells. Monocyte-derived dendritic cells (“moDCs”) were derived from PBMC by isolating monocytes with EasySep human monocytes Enrichment without CD16 depletion kit from Stemcell Technologies, followed by culturing 7 days in DCBM supplemented with GM-CSF (100 ng/ml) and IL-4 (50 ng/ml). cDC2 cells were differentiated from a TiPSC line (reprogrammed from the same donor PBMCs used to make the moDCs) using the conditions previously described with vitamin A and TNF on a DLL4-Fc-coated surface. cDC2 cells and moDC were either non-activated or activated with TNF/IL-1β/IL-6/prostaglandin E2 (“With Activation”) for 24 hours and washed three times prior to co-culture to remove activating compounds (FIG. 21A). Primary monocyte-derived DCs were CD209 (DC-SIGN)+, HLA class II+, CD11c+, CD1c^{low}, and CD14−. CD83 was strongly upregulated upon activation. Markers such as CD209 distinguish the moDCs as phenotypically different from the TiPSC-derived cDC2 cells. Allogenic primary T cells were isolated from PBMCs using EasySep human T cells isolation Kit from Stemcell Technologies to >90% purity. 2×10⁵ T cells were co-cultured in different ratios with allogenic TiPSC-cDC2 or moDCs for 6 days in T cell differentiation medium (TCDM) containing 2 ng/mL IL-2. T cell proliferation was measured as the total CD3+, CD4+, or CD8+ T cells number fold change compared to the starting population. All DCs stimulated robust T cell proliferation, with more potent stimulation from activated DCs. T cell stimulation by activated cDC2 was slightly superior to primary moDC. T cells without DCs (“None”), with anti-CD3/CD28 stimulating antibodies (0.5 μg/mL each) were included as controls. The same co-culture conditions were conducted at T:DC ratio of 5:1, with the exception that IL-2 was omitted from the TCDM (FIG. 21C). In the absence of IL-2, the TiPSC-derived cDC2 cells were more potent than primary MoDC at allogenic stimulation of primary T cells, particularly CD4+ T cells. T cells without DC (“None”), with anti-CD3/CD28 stimulating antibodies (0.5 μg/mL each) were included as controls. Anti-CD3/CD28 stimulation of T cells was poor in the absence of IL-2.

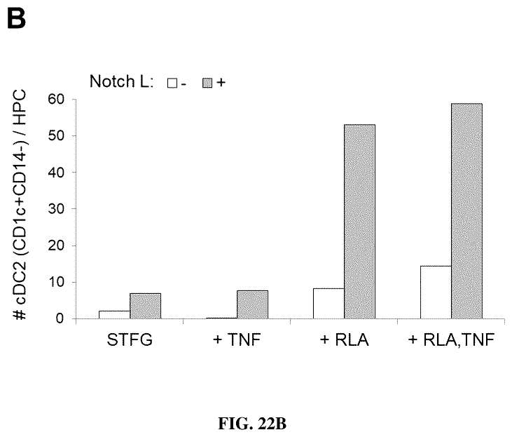

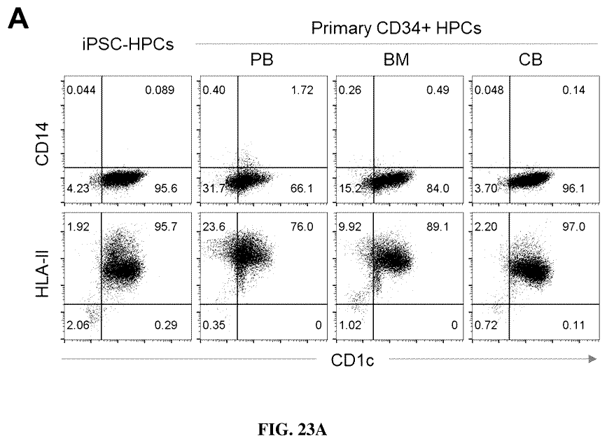

It was further shown that cDC2 could be efficiently differentiated from primary cord blood CD34+ HPCs using combination of cDC2 inductive factors Vitamin A, Notch ligand and TNF in FIG. 22. The identified cDC2 differentiation factors (e.g., Vitamin A, Notch L, and TNF) were tested to induce cDC2 from primary cord blood CD34+ HPCs. With the SCF, TPO, FLT3L, GM-CSF (STFG) basal condition, neither Vitamin A (RLA), TNF, or Notch L (DLL4) alone was sufficient to induce efficient cDC2 differentiation. The combination of Vitamin A (RLA) and Notch L (DLL4) had the highest suppression of CD14+ cells and direct differentiation to cDC2 lineage. Adding TNF resulted in ~90% pure CD1c+CD14− cDC2. Substantial cDC2 expansion in cultures was also observed with Vitamin A and Notch L combined and only slightly increased with added TNF. In contrast to iPSC-derived cDC2 cells, Notch activation was indispensable for cDC2 differentiation from primary HPCs. In addition, Vitamin A was needed, and TNF was an essential co-factor to produce primary cDC2 with high purity and yields.

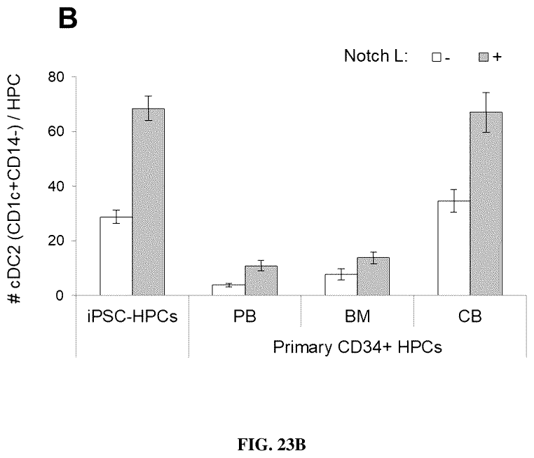

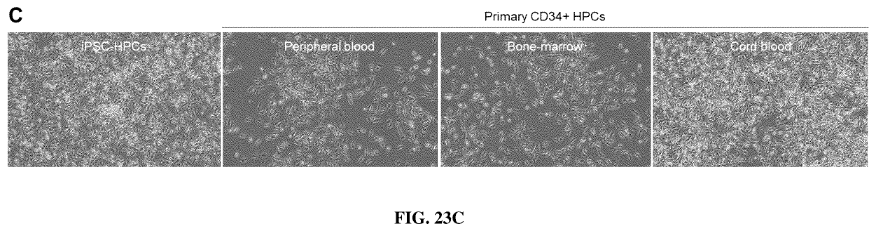

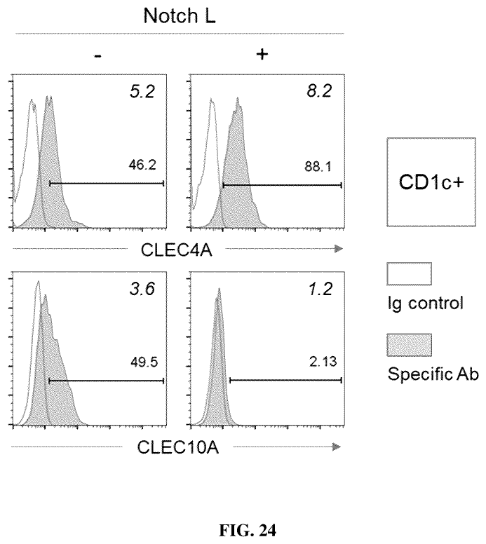

Finally, the cDC2 differentiation potential of iPSC-derived and primary CD34+ HPCs was determined (FIG. 23). cDC2 differentiation in iPSC-derived and primary CD34+ HPCs of embryonic (cord blood) and adult (peripheral blood, bone-marrow) origin was compared using combination of Notch L (DLL4-Fc, 0.25 μg/cm²), Vitamin A (water-soluble retinyl acetate, 2 μM, Sigma) and TNF (2 ng/mL) as cDC2 inductive factors. High purity CD1c+CD14− cDC2 cultures were derived from all HPC types. Lower CD1c expression was observed in adult HPC cultures, especially in those isolated from peripheral blood, however, >95% cells in all cultures expressed HLA class II (HLA-II) at high levels, suggesting cDC2 identity of CD1c^{low} cells. In terms of cDC2 yields, remarkably similar high yield proliferative cDC2 cultures were obtained with iPSC-derived and cord blood HPCs, while cDC2 yields in cultures from adult HPCs were ~6× lower due to much lower frequency of cDC2 differentiation initiating cell clusters in culture (visual observation). Nevertheless, after 6-day cDC2 differentiation, all cultures contained a morphologically identical population of small semi-adherent cells.

### Example 2—Materials and Methods

iPSCs derived cDC2 cells used in co-culture experiments: For the generation of cDC2 cells, iPSC-derived HPCs were resuspended and plated on Notch ligand-coated plates (DLL4-Fc, 0.1-1 μg/cm²) at 5×10³/cm² density for 6 days in DCDM medium supplemented with RLA (2 μM) SCF (50 ng/ml), FLT3L (50 ng/ml), GM-CSF (50 ng/ml), and TNF (2 ng/ml). Cells were fed every 2-3 days. On day 6, cDC2 cells were washed with DPBS and seeded with or without activation with soluble CD40L (50 ng/ml) or TB6P (10 ng/ml TNF/IL-1β/IL-6, 200 ng/ml ProstaglandinE2) in DCDM without RLA and TNF for one day. Semi-adherent cDC2 cells were harvested with 0.5 mM EDTA/PBS and washed three times with DPBS to avoid any residue medium carry over from DC differentiation. cDC2 cells were resuspended in “primary T cell medium” (CTS AIM-V Medium supplemented with 2% human AB serum, 1× Glutamax supplement, 250 μM L-ascorbic acid phosphate magnesium salt n-hydrate, and 100 μM monothioglycerol) for flow cytometry analysis and co-culture experiments.

Primary monocyte-derived dendritic cells (moDCs): PBMCs (AllCells) were thawed and cultured in DCDM medium overnight. Monocytes were isolated from the PBMCs using the EasySep Human Monocyte Enrichment Without CD16 Depletion Kit (Stemcell Technologies) according to the manufacturer's instructions and then cultured in DCDM medium with GM-CSF (100 ng/ml) and IL-4 (50 ng/ml) for 7 to 8 days, changing medium every 2 days and carefully removing cells in suspension during the first two medium exchanges. To activate the moDC, TB6P (10 ng/ml TNF, IL-1p, and IL-6, and 200 ng/ml prostaglandin E2) was added to the culture medium for the last 24 hours. MoDCs were harvested using TrypLE Select (Gibco) and washed for flow cytometry analysis and co-culture experiments.

Allogenic primary T cell preparation: PBMCs (AllCells) were thawed and cultured in AIM-V medium overnight to recover from cryopreservation. The cells were then treated with DNase I solution (100 μg/mL, Stemcell Technologies) for 15 min at room temperature, washed, and run through a 30 μm MACS SmartStrainers (Miltenyi Biotec). T cells were isolated using the EasySep human T cell Isolation Kit (StemCell Technologies) according to the manufacturer's instructions to remove APCs and other non-T cells. T cells were labeled with 1-10 μM CellTrace CFSE dye (Thermofisher) for 5-20 min, washed, and resuspended in primary T cell medium for co-culture experiments.

cDC2 or primary moDCs co-culture with allogenic primary T cells: CFSE-labeled T cells (2×10⁵/well) were co-cultured with different numbers of iPSC-derived cDC2 cells or primary moDC, with ratios ranging from 5:1 to 320:1 in primary T cell medium with or without IL-2 (2 ng/ml). Negative and positive control conditions were T cells alone or T cells stimulated with anti-CD3/CD28 antibodies (0.5 μg/ml each), respectively. Half medium exchange was performed every other day. After 6 days, the cells were harvested for cell counts and flow cytometry. The amount of proliferation was calculated using cell counts corrected with the CD3/CD4/CD8 subset percentages before and after co-culture and is reported as cell number relative to the starting population (x-fold expansion).

Antigen specific T cell isolation and reprogramming to iPSCs: Peripheral blood mononuclear cells (PBMCs) from healthy donors with HLA DRB1*01:01 haplotype were resuspended at 3×10⁶ cells/mL in RPMI T cell Medium (RPMI with 10% AB human serum, 25 mM HEPES, 1 mM sodium pyruvate, and 1× GlutaMAX) containing Influenza HA (306-318) peptide (10 μg/mL, MBL Life Science). Cell were cultured for 5-7 days (5% CO₂, 37° C.) and then fed (50% exchange) with fresh medium containing IL-2 (1 ng/ml). Cell were fed and/or split on subsequent days based on confluence. On day 16, the cells were replated and re-stimulated using feeder cells. Feeder cells were derived from autologous PBMCs metabolically inactivated with mitomycin C (20 μg/mL for 3 hours), pulsed with HA peptide (10 μg/mL for 2-3 h), followed by washing three times with DPBS to remove mitomycin C. Restimulation culture was for an additional 14 days, fed as described above. The emergence of antigen-specific CD4+ T cells could be detected using flow cytometry and a fluorophore-labeled HLA-DRB1*01:01/influenza HA (306-318) tetramer reagent (MBL LIfe Science, hereafter “HA tetramer”).

HA-specific T cells were isolated using Flow Activated Cell Sorting (FACS), sorting on CD4+HA Tetramer+ cells to 99% purity. Sorted cells were restimulated with peptide-pulsed feeder cells as described above. The cells were fed and expanded as described above for 11 days, with additional feeder cells added on day 5. On day 11, cells were restimulated with a combination of peptide-pulsed feeder cells and anti-CD3/CD28 Dynabeads (Thermofisher) and cultured for an additional 3 days.

T cells were reprogrammed to TiPSC using the CytoTune-iPS 2 0 Sendai Reprogramming Kit (Thermofisher) following the manufacturer's protocol. Individual TiPSC colonies were picked, transitioned to Essential 8 medium on vitronectin ECM surface, and cultured for approximately 20 passages to ensure loss of reprogramming virus plasmid. Plasmid loss was confirmed using quantitative PCR primers for SEV-KOS, SEV-CMYC, and SEV-KLF4 (Thermofisher).

To screen for TiPSC clones with rearranged TCR loci specific for the HA (306-318) antigen, TiPSC lines were differentiated to T cells. Differentiation to CD34⁺CD43⁺CD45+ HPC and CD3+ T cells was performed using procedures previously described (U.S. Pat. No. 10,947,502, incorporated by reference herein in its entirety), with minor modifications. Briefly, HPC were plated on tissue culture plates pre-coated with recombinant Delta-like protein 4 (DLL4; 0.5 μg/cm²) and RetroNectin (0.5 μg/cm²) for 14 days in T cell Differentiation Medium (TCDM), with medium feed (50% exchange) every 2-3 days. TCDM medium is composed of CTS AIM V Medium supplemented with CTS Immune Cell Serum Replacement (10%), 1× Glutamax supplement, L-ascorbic acid phosphate magnesium salt n-hydrate (250 μM), monothioglycerol (100 μM), nicotinamide (2 mM), SCF (50 ng/ml), FLT3L (50 ng/ml), TPO (50 ng/ml), and IL-7 (50 ng/ml). T cells expressing the HLA-DRB1*01:01-HA peptide-specific TCR were identified by binding of the HA Tetramer reagent using flow cytometry. TiPSC clone/“Line 2” was selected for co-culture experiments. The iT cells were either cryopreserved or further cultured on fresh DLL4 (0.5 μg/cm²) and RetroNectin (0.5 μg/cm²) coated surface in TCDM medium. T cells differentiated for 14-21 day (post-HPC) were used for autologous cDC2 co-culture experiments.

cDC2 co-culture with autologous antigen-specific iT cells: Antigen-specific TiPSC-derived T cells (iT) were differentiated and CFSE-labeled as described above. Autologous cDC2 cells—derived from the same TiPSC line as the iT cells—were differentiated and activated as described above. The cDC2 cells were pulsed with HA peptide or CLIP irrelevant peptide (1 μg/ml, MBL Life Science). CFSE-labeled iT cells (2×10⁵/well) were co-cultured with different numbers of iPSC-derived cDC2 cells with ratios ranging from 5:1 to 320:1 in TCDM with IL-2 (1 ng/ml). Negative and positive control conditions were iT cells alone or iT cells stimulated with anti-CD3/CD28 antibodies (0.5 μg/ml each), respectively. Half medium exchange was performed every other day. After 7 days, the cells were harvested for cell counts and flow cytometry. The amount of proliferation was calculated using cell counts, confirming the T cell phenotype (CD3+) of the proliferating (CFSE-reduced) cells, and is reported as T cell number relative to the starting population (x-fold expansion).

cDC2 phagocytosis activity: pHrodo Green Zymosan A Bioparticles Conjugate (S. cerevisiae, Thermofisher) was resuspended for use in HBSS (5 mg/ml). A 10 mM stock (5 mg/ml, 1000× final concentration) of cytochalasin D (Sigma) was prepared in DMSO. cDC2 cells (105 per tube) were pretreated with cytochalasin D (10 μg/ml) or DMSO (control) for 15 min. Cells were washed, and pHrodo Green Zymosan A Bioparticles were added (10 μg/ml) to cDC2 cells in DCDM without RLA or TNF, and incubated at 37° C. (5% CO₂ incubator) or 0° C. (on ice) for 1 hour. Live dead staining was performed with propidium iodide (PI, 1 μg/ml) prior to flow cytometry.

cDC2 receptor mediated endocytosis activity: DQ-ovalbumin (Thermofisher) stock was prepared in DPBS (2 mg/ml). DQ-ovalbumin was added (10 μg/tube) to cDC2 cells (105 per tube) in DCDM without RLA or TNF, and incubated at 37° C. (5% CO₂ incubator) or 0° C. (on ice) for 45 minutes. Live dead staining was performed with propidium iodide (PI, 1 μg/ml) prior to flow cytometry.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Amit et al., Dev. Bio., 227:271-278, 2000.
Byrne et al., Nature, 450(7169):497-502, 2007.
Fernandes, et al., J. Biotechnology, 132(2):227-236, 2007.
International Patent Publication No. PCT/JP2009/062911
International Patent Publication No. PCT/JP2011/069588
International Patent Publication No. WO 02/44343
International Patent Publication No. WO 03/050251
International Patent Publication No. WO 2007/069666
International Patent Publication No. WO2005/123902
International Patent Publication No. WO98/30679
Keller et al., Curr. Opin. Cell Biol., 7:862-869, 1995.
Klimanskaya et al., Lancet., 365:P1636-1641, 2005.
Ludwig et al., Nat. Biotechnol., 24:185-187, 2006b.
Ludwig et al., Nat. Methods, 3:637-646, 2006a.
Takahashi et al., Cell, 131, 861-872, 2007.
Thomson and Marshall, Curr. Top. Dev. Biol., 38:133-165, 1998.
Thomson and Odorico, Trends Biotechnol., 18(2):53-57, 2000.
Thomson et al. Proc. Natl. Acad. Scie. USA, 92:7844-7848, 1995.
U.S. application Ser. No. 12/478,154
U.S. Pat. No. 8,546,140
U.S. Pat. No. 5,843,780
U.S. Pat. No. 6,103,470
U.S. Pat. No. 6,110,739
U.S. Pat. No. 6,200,806
U.S. Pat. No. 6,280,718
U.S. Pat. No. 6,416,998
U.S. Pat. No. 7,029,913
U.S. Pat. No. 7,442,548
U.S. Pat. No. 7,598,364
U.S. Pat. No. 7,989,425
U.S. Pat. No. 8,058,065
U.S. Pat. No. 8,071,369
U.S. Pat. No. 8,129,187
U.S. Pat. No. 8,183,038
U.S. Pat. No. 8,268,620
U.S. Pat. No. 8,546,140
U.S. Pat. No. 8,691,574
U.S. Pat. No. 8,741,648
U.S. Pat. No. 8,900,871
U.S. Pat. No. 9,175,268
U.S. Patent Publication No. 2003/0211603
U.S. Patent Publication No. 2009/0246875
U.S. Patent Publication No. 2010/0210014
U.S. Patent Publication No. 2011/0104125
U.S. Patent Publication No. 2012/0276636
U.S. Patent Publication No. 2015/0191697
U.S. Patent Publication No. US 2002/0086005
United States Patent 20070116680
Watanabe et al., Nature Neurosci., 8:288-296, 2005.
Xu et al., Nat. Biotechnol., 19:971-974, 2001.
Yu et al., Science, 318: 1917-1920, 2007.

1. A method for producing conventional type 2 dendritic cells (cDC2) from hematopoietic progenitor cells (HPCs) comprising:
(a) obtaining a population of HPCs; and
(b) differentiating the HPCs in dendritic cell differentiation media comprising a retinoid signaling agonist to produce a population of cDC2.

2. (canceled)

3. (canceled)

4. The method of claim 1, wherein the population of HPCs comprises cells positive for CD34, CD43, and/or CD45.

5. (canceled)

6. The method of claim 1, wherein the media of step (b) further comprises a Notch activator.

7. The method of claim 6, wherein the Notch activator is selected from the group consisting of DLL4, DLL1, JAG1, and JAG2.

8. (canceled)

9. The method of claim 1, wherein the media of step (b) further comprises at least one pro-inflammatory cytokine and/or dendritic cell growth factor.

10. The method of claim 9, wherein the at least one pro-inflammatory cytokine is selected from the group consisting of TNF, IL-1β, lymphotoxin and IL-1α and/or the at least one dendritic cell growth factor is selected from the group consisting of GM-CSF, IL-3, and M-CSF.

11. (canceled)

12. (canceled)

13. (canceled)

14. The method of claim 1, wherein the retinoid signaling agonist is a naturally occurring biologically active retinoid or a synthetic agonist of retinoic acid receptor (RAR).

15. The method of claim 14, wherein the retinoid is vitamin A.

16. The method of claim 15, wherein the vitamin A is retinyl acetate, retinol, retinyl palmitate, all-trans retinoic acid, or 9-cis retinoic acid.

17.-22. (canceled)

23. The method of claim 14, wherein the synthetic agonist of RAR is TTNPB or AM580.

24. The method of claim 1, wherein the media of step (b) further comprises at least one hematopoietic growth factor.

25. The method of claim 24, wherein the at least one hematopoietic growth factor is selected from the group consisting of SCF, FLT3L, and TPO.

26. (canceled)

27. The method of claim 1, wherein differentiating comprises culturing HPC in media comprising a Notch activator, pro-inflammatory cytokine, dendritic cell growth factor, and/or hematopoietic growth factor.

28. The method of claim 1, wherein step (b) comprises differentiating CD34⁺CD45⁺ HPCs in media comprising vitamin A, a Notch activator, GM-CSF, TNF, and SCF to produce a population of cDC2.

29. The method of claim 1, wherein the population of cDC2 comprise at least 90% CD1c⁺/CD14⁻ cells and/or the method produces more than 25 cDC2 per input HPC.

30.-41. (canceled)

42. The method of claim 1, wherein the population of HPCs are further defined as a population of primary HPCs.

43.-46. (canceled)

47. The method of claim 1, wherein the CD1c⁺/CD14⁻ cDC2 further express one or more of CD11c, HLA class II, CD80, CD86, CD172a/SIRPα, CLEC4A, and CD11b.

48.-49. (canceled)

50. The method of claim 1, wherein the CD1c⁺/CD14⁻ cDC2 have increased expression of IRF4, CD1C, ITGAX, CLEC4A, TLR2, TLR7, and TLR10 and/or CLEC10A as compared to the HPCs.

51.-57. (canceled)

58. The method of claim 1, further comprising activating the population of cDC2 to acquire a mature phenotype in activated cDC2, wherein the activated cDC2 have increased expression of HLA class II and co-stimulatory receptors CD80, CD86, and CD40 as compared to the non-activated resting cDC2.

59.-62. (canceled)

63. The method of claim 1, wherein the cDC2 trigger proliferation of T cells in a co-culture with T cells.

64.-74. (canceled)

75. A composition comprising at least 80% CD1c⁺/CD14⁻ cDC2 produced by the methods of claim 1.

76.-83. (canceled)

84. A method of stimulating T cells comprising culturing said T cells with a composition of cDC2 of claim 1.

85.-140. (canceled)
